(19) United States
(12) Patent Application Publication
(10) Pub. No.: US 2014/0371633 A1
(43) Pub. Date: Dec. 18, 2014

(54) METHOD AND SYSTEM FOR EVALUATING A PATIENT DURING A REHABILITATION EXERCISE

(71) Applicant: JINTRONIX, INC., WILMINGTON, DE (US)

(72) Inventors: Mark Evin, Montreal (CA); Ofer Allan Avital, Montreal (CA); Justin Tan, Montreal (CA); Alexis Youssef, Westmount (CA); Sung Jun Bae, Montreal (CA)

(21) Appl. No.: 14/364,351

(22) PCT Filed: Dec. 13, 2012
(86) PCT No.: PCT/US12/69482

§ 371 Date: Jun. 11, 2014

Related U.S. Application Data
(60) Provisional application No. 61/576,092, filed on Dec. 15, 2011.

(51) Int. Cl. A61B 5/11; A61B 5/16

(52) U.S. Cl. CPC A61B 5/1128; A61B 5/1102; A61B 5/16
USPC 600/595

(57) ABSTRACT

A method for evaluating a user during a virtual-reality rehabilitation exercise, comprising: receiving a target sequence of movements comprising at least a first target elementary movement and a second target elementary movement; receiving a measurement of a movement executed by the user while performing the rehabilitation exercise and interacting with a virtual-reality simulation comprising at least a virtual user-controlled object, a characteristic of the virtual user-controlled object being controlled by the movement; determining, from the measurement of the movement executed by the user, a sequence of measured movements comprising at least a first measured elementary movement and a second measured elementary movement; comparing the sequence of measured movements to the sequence of target movements; and outputting an evaluation.

### CROSS REFERENCE TO RELATED APPLICATIONS

This application claims priority of U.S. Provisional Patent Application having Ser. No. 61/576,092, which was filed on Dec. 15, 2011 and is entitled “VIRTUAL REALITY REHABILITATION SYSTEM”, the specification of which is hereby incorporated by reference.

### TECHNICAL FIELD

The present subject matter relates to systems for physical rehabilitation of patients, and more particularly methods and systems for evaluating a patient during a rehabilitation exercise.

### BACKGROUND

Many people worldwide suffer from motor, cognitive, sensory, and musculoskeletal disorders. Rehabilitation is usually necessary to help return these people back to a functional state.

Rehabilitation exercises are usually used for retraining neural pathways or training new neural pathways to regain or improve neurocognitive functioning that has been diminished by disease or traumatic injury such as a stroke for example. Such exercises are usually performed under the supervision of a medical professional such a therapist or a clinician in a hospital or a rehabilitation center. Due to the cost associated with rehabilitation, hospitals and rehabilitation centers may not have enough space and/or personnel to accommodate every patient's needs. Therefore, medical professionals usually prescribe to patients rehabilitation exercises to be performed at home. However, these rehabilitation exercises are not performed under the supervision of a medical professional who cannot evaluate whether the patient has adequately performed the rehabilitation exercise. In addition, even when a medical professional supervises a rehabilitation exercise performed by a patient, it may be difficult for the medical professional to analyze all parameters and estimate whether the patient executes adequately the rehabilitation exercise.

Therefore, there is a need for an improved method and system for evaluating the performance of a patient during a rehabilitation exercise.

### SUMMARY

In accordance with a first broad aspect, there is provided a computer-implemented method for evaluating a user during a virtual-reality rehabilitation exercise, comprising: receiving a target sequence of movements comprising at least a first target elementary movement and a second target elementary movement, the first target elementary movement defined by a first body part and a first movement type and the second target elementary movement defined by a second body part and a second movement type, the first and second target elementary movements being different; receiving a measurement of a movement executed by the user while performing the rehabilitation exercise and interacting with a virtual-reality simulation comprising at least a virtual user-controlled object, a characteristic of the virtual user-controlled object being controlled by the movement; determining, from the measurement of the movement executed by the user, a sequence of measured movements comprising at least a first measured elementary movement and a second measured elementary movement; comparing the sequence of measured movements to the sequence of target movements, thereby obtaining an evaluation of a performance of the user; and outputting the evaluation.

In one embodiment, the step of receiving a target sequence of movements further comprises receiving a first target range of movement for the first target elementary movement and a second target range of movement for the second target elementary movement, and the step of determining a sequence of measured movements further comprises determining a first measured range of movement for the first target elementary movement and a second measured range of movement for the second target elementary movement, at least one of the first and second target elementary movements being different and the first and second target ranges of movement being different.

In one embodiment, the step of receiving the measurement of the movement executed by the user comprises receiving the first measured range of movement for the first measured elementary movement and the second measured range of movement for the second measured elementary movement, and the step of determining the sequence of measured movements comprises temporally ordering the first and second elementary movements.

In another embodiment, the step of receiving the measurement of the movement executed by the user comprises receiving position information for the first body part and the second body part.

In one embodiment, the step of receiving position information comprises, for each one of the first and second body parts, receiving at least one of an angle in time and a position in time of reference points.

In one embodiment, the step of determining a sequence of measured elementary movements comprises: determining, for each one of the first and second body parts, a type of movement, thereby identifying the first and second measured elementary movements; determining the first and second measured range of movements from the position information; and determining an order of execution for the first and second measured elementary movements, thereby obtaining the sequence of measured elementary movements.

In one embodiment, the step of determining a type of movement comprises: determining a movement axis from the position information; and assigning the type of movement as a function of the movement axis.

In one embodiment, the step of determining a type of movement further comprises determining a movement direction using the position information, said assigning the type of movement comprising assigning the type of movement as a function of the movement axis and the movement direction.

In one embodiment, the step of comparing comprises: comparing the first and second measured elementary movements to the first and second target elementary movements, respectively; and comparing the first and second measured ranges of movement to the first and second target ranges of movement, respectively.

In one embodiment, the step of outputting an evaluation comprises outputting an indication as to whether the user failed to execute at least one of the first and second target elementary movement.

In accordance with another broad aspect, there is provided a system for evaluating a user during a virtual-reality rehabilitation exercise, comprising: a communication unit for: receiving a target sequence of movements comprising at least a first target elementary movement and a second target elementary movement, the first target elementary movement defined by a first body part and a first movement type and the second target elementary movement defined by a second body part and a second movement type, the first and second target elementary movements being different; receiving a measurement of a movement executed by the user while performing the rehabilitation exercise and interacting with a virtual-reality simulation comprising at least a virtual user-controlled object, a characteristic of the virtual user-controlled object being controlled by the movement; and outputting an evaluation of a performance of the user; a sequence determining unit for determining, from the measurement of the movement executed by the user, a sequence of measured movements comprising at least a first measured elementary movement and a second measured elementary movement; and a comparison unit for comparing the sequence of measured movements to the sequence of target movements in order to obtain the evaluation of the performance of the user.

In one embodiment, the communication unit is further adapted to receive a first target range of movement for the first target elementary movement and a second target range of movement for the second target elementary movement, and the sequence determining unit is further adapted to determine a first measured range of movement for the first target elementary movement and a second measured range of movement for the second target elementary movement, at least one of the first and second target elementary movements being different and the first and second target ranges of movement being different.

In one embodiment, the measurement of the movement executed by the user comprises the first measured range of movement for the first measured elementary movement and the second measured range of movement for the second measured elementary movement, the sequence determining unit is adapted to temporally order the first and second elementary movements.

In another embodiment, the measurement of the movement executed by the user comprises position information for the first body part and the second body part.

In one embodiment, the position information comprises, for each one of the first and second body parts, at least one of an angle in time and a position in time of reference points.

In one embodiment, the sequence determining unit is adapted to: determine, for each one of the first and second body parts, a type of movement, thereby identifying the first and second measured elementary movements; determine the first and second measured range of movements from the position information; and determine an order of execution for the first and second measured elementary movements in order to obtain the sequence of measured elementary movements.

In one embodiment, the sequence determining unit is further adapted to: determine a movement axis from the position information; and assign the type of movement as a function of the movement axis.

In one embodiment, the sequence determining unit is further adapted to determine a movement direction from the position information and assign the type of movement as a function of the movement axis and the movement direction.

In one embodiment, the comparison unit is adapted to: compare the first and second measured elementary movements to the first and second target elementary movements, respectively; and compare the first and second measured ranges of movement to the first and second target ranges of movement, respectively.

In one embodiment, the evaluation comprises an indication as to whether the user failed to execute at least one of the first and second target elementary movement.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

In order that the invention may be readily understood, embodiments of the invention are illustrated by way of example in the accompanying drawings.

FIG. 1 is a flow chart illustrating a method for evaluating a user during a rehabilitation exercise, in accordance with an embodiment;

FIGS. 2a-2c illustrate a simulation to be provided to a user during a rehabilitation exercise, in accordance with an embodiment;

FIG. 3 is a block diagram illustrating a virtual reality rehabilitation system, in accordance with a first embodiment;

FIG. 4 is a block diagram illustrating a virtual reality rehabilitation system, in accordance with a second embodiment;

FIG. 5 illustrates a table presenting activity specific data, in accordance with an embodiment;

FIG. 6 is a graph presenting an evolution of a activity-specific data over time, in accordance with an embodiment;

FIG. 7 illustrate an interface to be presented to a medical professional for creating a virtual rehabilitation exercise, in accordance with an embodiment;

FIG. 8 is a block diagram illustrating a system for generating a virtual reality simulation for a user and generating an evaluation report, in accordance with an embodiment;

FIG. 9 is a flow chart of method for evaluating a user performance during a virtual reality rehabilitation, in accordance with an embodiment;

FIG. 10 illustrates a trunk rotation movement for a user;

FIG. 11 illustrates a shoulder abduction movement for a user;

FIG. 12 illustrates a virtual rehabilitation exercise in which two plates have to be brought together to catch a falling object, in accordance with an embodiment;

FIG. 13 illustrates a virtual rehabilitation exercise in which a user has to move his trunk to hit a target, in accordance with an embodiment; and

FIG. 14 illustrates a virtual rehabilitation exercise in which a user has to move a hand to control a fish, in accordance with an embodiment.

It will be noted that throughout the appended drawings, like features are identified by like reference numerals.

Further details of the invention and its advantages will be apparent from the detailed description included below.

### DETAILED DESCRIPTION

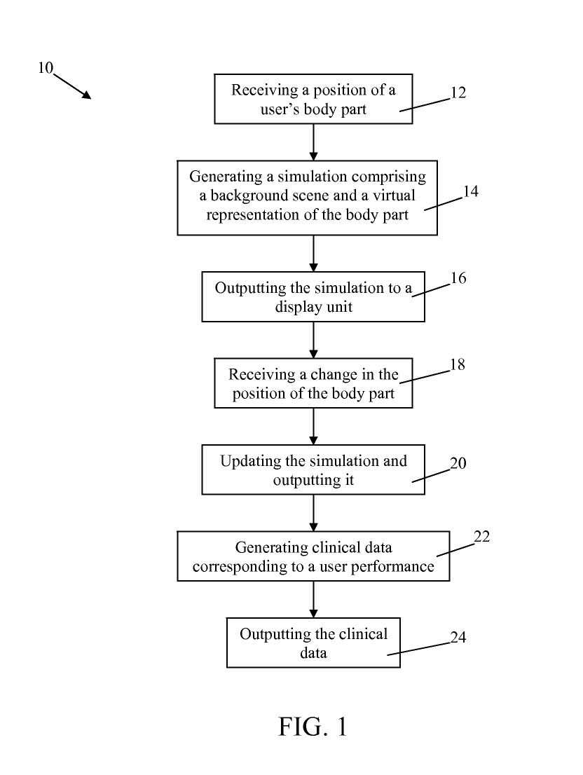

FIG. 1 illustrates one embodiment of a computer-implemented method 10 for providing a user or patient with rehabilitation exercises within a virtual reality environment. The method 10 allows for rehabilitation of a body part of a user/patient. For example, the method 10 may be used for upper-body rehabilitation, i.e. the rehabilitation of a hand, fingers, an arm, a wrist, an elbow, a shoulder, a trunk, and/or the like. In another example, the method 10 may be used for lower-body rehabilitation, such as rehabilitation of a foot, a knee, or the like.

While FIG. 1 is described below with respect to the rehabilitation of a hand, it should be understood that the method 10 may be performed for the rehabilitation of any adequate body part or limb.

The first step 12 of the method comprises receiving a position of the user's hand within a three-dimensional (3D) space from a motion tracking unit or motion sensing device. The position of the hand is received substantially continuously during the execution of the rehabilitation exercise by the user. In one embodiment, the step 12 further comprises receiving the orientation of the hand and/or the flexion/extension state of at least one finger or digit, i.e. the position of the finger(s) with respect to the hand palm.

At step 14, a simulation comprising an interactive environment formed of a background scene and a virtual representation of the hand is generated. The simulation is to be displayed to the user and provides the user with a virtual environment in which the user will execute a rehabilitation exercise. Therefore, each simulation is associated with a respective rehabilitation exercise to be executed by the user. In one embodiment, the background scene is a 2D or 3D computer graphic or image to be displayed to the user. The position of the virtual representation of the hand within the background scene is set as a function of the received hand position, i.e. the position of the hand within the 3D space.

In one embodiment, the virtual representation of the hand is hand-shaped. i.e. it mimics the shape of a human hand. In another embodiment, the virtual representation of the hand may be an object, an animal, a dot, and or the like.

In one embodiment, the background scene may only comprise a background image having fixed virtual elements, i.e. none of the elements constituting the background scene may be moved, while the virtual representation of the hand may move within the background scene. For example, the background scene may comprise a first and a second reference marks or points which have a fixed position.

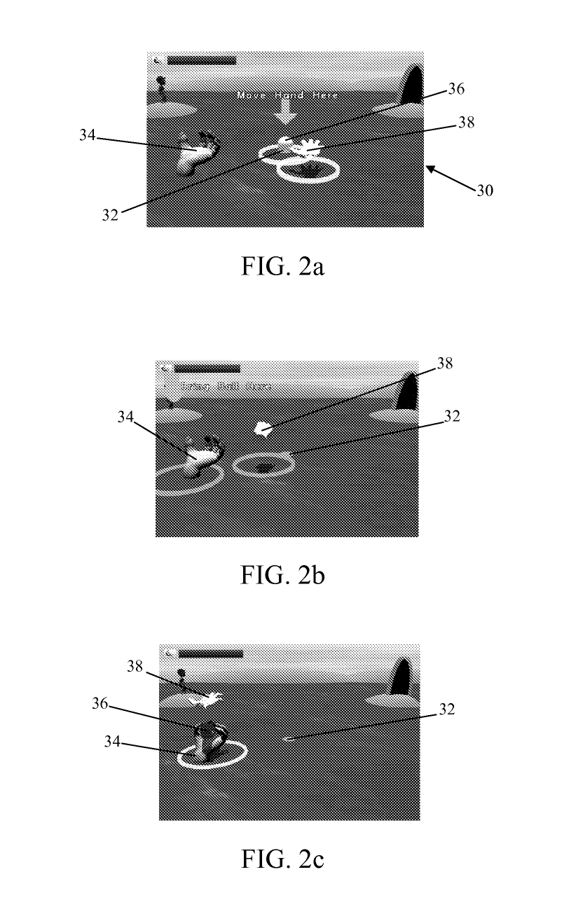

In another embodiment, the background scene may comprise a background image and at least one virtual element that may be moved with respect to the background image. For example, FIG. 2a illustrates one embodiment of a background scene 30 comprising a reference mark 32 and a ball receiving hand 34, which both have a fixed position within the background scene 30. The background scene 30 further comprises a virtual ball 36 which may be moved within the background scene 30. The simulation further comprises a virtual representation 38 of the hand of the user. The virtual hand 38 is positioned within the background scene 30 according to the position of the user hand within the 3D space.

It should be understood that the background image may be any adequate image such as an image of a scenery, a single-color font, etc.

At step 16, the simulation is output to a display unit for being displayed to the user on a display unit so that the user may watch the virtual representation of his hand within the background scene on the display unit.

The user is provided with instructions for executing at least one task within the virtual environment in order to execute a rehabilitation exercise. The task may correspond to a single elementary movement or a sequence of elementary movements. For example, the user may be instructed to raise his hand, to open his hand, to bring a ball to a target position, and/or the like. The instructions may be provided before the start of the simulation, or during the simulation as illustrated in FIGS. 2a and 2b. The instructions may be vocal instructions, visual instructions, etc. The instructions may be independent from the simulation. For example, the instructions may be sent to the user via an email independently from the simulation so that the user may read the instructions before starting the simulation and executing the rehabilitation exercise. In another example, the instructions may be integrated within the simulation. For example, the instructions may comprise written instructions displayed within the interactive environment. In another example, the instructions may comprise a virtual element, such as an arrow, which is integrated within the interactive environment.

Referring back to the first example in which the background scene comprises the first and second reference marks or points, the user may be instructed to move his hand from the first reference mark to the second reference mark before the start of the simulation for example. Referring back to the example illustrated in FIG. 2a, a written instruction “Move Hand Here” is inserted in the simulation to be displayed to the user. Alternatively, vocal instructions could be provided to the user during the simulation.

Following the instructions, the user moves his hand in the 3D space. A hand movement should be understood as any movement of the hand within the 3D space such as a translation of the hand, a rotation of the hand, a flexion or extension of at least one finger, and/or the like. A movement of the hand may be caused by a translation and/or rotation of the wrist, elbow, and/or shoulder, and/or a flexion or extension of a finger. The 3D tracking system detects the hand movement and transmits the new position of the hand so that the change in the position of the hand is received at step 18.

At step 20, the simulation is updated according to the new position of the hand within the 3D space, and output to the display unit. The position of the virtual representation of the hand within the background scene is modified according to the change in position of the hand within the 3D space. As a result, when he moves his hand in the 3D space, the user sees a corresponding movement of the virtual hand within the background scene on the display unit.

Referring back to the first example in which the user is instructed to move his hand from the first reference mark to the second reference mark, the user follows the instructions and sees in substantially real-time the virtual representation of his hand within the background scene following the same movement as that of his hand in the 3D space.

Referring to FIGS. 2a-2c, the user moves his hand so that the virtual representation 38 of his hand be vertically aligned with the virtual hall 36 and then closes his hand in the 3D space so that the virtual representation 38 of his hand grasps the virtual ball 36. Then the user is instructed to bring the virtual ball 36 up to the virtual ball receiving hand 34. The user moves his hand in the 3D space while maintaining his grasp hand position so that the virtual representation 38 of his hand be positioned on top of the virtual ball receiving hand 34 while still grasping the virtual ball 36. The user then opens his hand in the 3D space so that the virtual representation 38 of his hand releases the virtual ball 36 in the virtual ball receiving hand 34.

In one embodiment, the position of the hand within the 3D space is received substantially continuously during the simulation, i.e. during the execution of the rehabilitation exercise by the user. For example, the motion tracking unit may send continuously the position of the user's hand within the 3D space. In another example, the position of the user's hand may be sent periodically at predetermined time intervals. The time intervals may be chosen to be short enough for providing the user with a real-time experience.

In one embodiment, the received position of the hand is stored in memory during the simulation. The continuous position of the hand may be recorded or only the positions of the hand at predetermined time intervals may be recorded.

At step 22, activity-specific data are generated and then output at step 24. The activity-specific data measure the performance of the user during the simulation, i.e. they provide the user with at least one score for the rehabilitation exercise. The activity-specific data are generated from the interaction of the user with the simulation. In one embodiment, at least some activity-specific data may be extracted from the simulation and used by a medical professional to evaluate the user performance during the execution during the rehabilitation exercise. In the same or another embodiment, at least some activity-specific data may also be extracted from the position data provided by the motion tracking unit.

In one embodiment, the activity-specific data comprise the speed of movement, which is determined from the time taken by the user to move his hand between two reference points and the distance between the two reference points.

In one embodiment, the activity-specific data comprise the accuracy of movement. The accuracy of movement is determined from the position of the virtual representation of the hand within the background scene during the simulation, i.e. the recorded position of the hand within the 3D space during the simulation. For example, the accuracy of movement may be determined from the deviation of the path followed by the virtual representation of the hand from the shortest distance straight line path between two reference points.

In one embodiment, the activity-specific data comprise the number of failed and/or successful attempts in achieving a particular task during the simulation. For example, during the simulation the user may be asked to grasp and lift a virtual ball and then release the ball at a specific reference point. Fail attempts could correspond to the following scenarios: the user cannot grasp the ball, the user releases the ball before reaching the reference point, etc.

In one embodiment, the activity-specific data comprise the finger flexion/extension range for at least one finger.

In one embodiment, the activity-specific data comprise the time required by the user to complete a particular task during the simulation and/or the whole simulation. For example, the activity-specific data may comprise the time taken by the user for grasping the virtual ball starting from the beginning of the simulation, i.e. the beginning of the rehabilitation exercise. In another example, the activity-specific data may comprise the time required by the user to move his hand between two virtual reference points.

In one embodiment, the activity-specific data comprise the release and/or grasp accuracy. For example, the release accuracy may correspond to the distance between the center of a target mark on which the user has to release a virtual ball and the actual point at which the user released the virtual ball. In one embodiment, the grasp accuracy comprises two measurements. The first measurement consists in the distance between the 3D position of the user's virtual hand at the point at which the user closes grasp, and the ideal 3D position of the user's hand should be at in order to correctly grasp on object. The smaller the distance, the better the grasp accuracy. The second measurement consists in the actual amount that the user is able to close his grasp. For example, if a user is able to fully close his grasp, that would be measured as high grasp accuracy.

In one embodiment, the simulation which is displayed to the user may be recorded and the activity-specific data may comprise a video of the simulation. In another embodiment, the activity-specific data may comprise activity data points from which the simulation may be subsequently re-rendered.

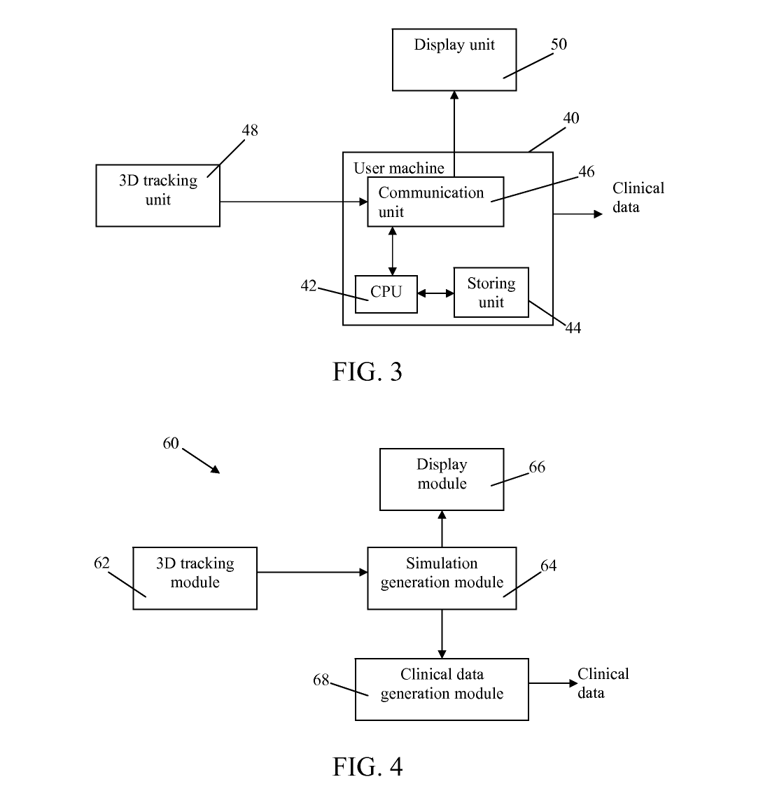

It should be understood that the method 10 may be embodied in an apparatus or machine 40 adapted to perform the steps of the method 10, as illustrated in FIG. 3. The machine 40 is provided with a processing unit 42 such as a Central Processing Unit (CPU) connected to a storing unit 44. The storing unit 44 may be any adequate device for storing digital data, such as a hard drive, a flash memory, and the like. The storing unit 44 may be integrated into the machine 40 or external to the machine 40. The processing unit 42 is configured for performing the steps of the method 10. The machine 40 further comprises a communication unit 46 connectable to a motion tracking unit 48 for receiving the position of the hand within the 3D space, and a display unit 50. The simulation is sent to the display unit 50 via the communication unit 48 to be displayed to the user in substantially real-time.

The method 10 may also be embodied in a system 60 as illustrated in FIG. 4. The system 60 comprises a motion tracking module 62, a simulation generation module 64, a display module 66, and an activity-specific data generation module 68. The motion tracking module 62 is adapted to substantially continuously track and determine the position of at least the hand of the user in the 3D space. It should be understood that the motion tracking module 62 may also be adapted to determine the orientation of the hand in the 3D space and the flexion state of at least one finger of the hand. In one embodiment, the motion tracking module 62 is adapted to determine the flexion/extension state of each finger independently from that of the others. In another embodiment, the flexion/extension state of only one reference finger is determined and the flexion/extension state of the other fingers is determined according to that of the reference finger.

The simulation generation module 64 receives substantially continuously the position of the hand within the 3D space from the motion tracking module 62 and is adapted to generate a simulation comprising a background scene and a virtual representation of the hand, as described above. The position of the virtual representation of the hand within the background scene is set as a function of the received position of the hand within the 3D space.

In one embodiment, the simulation generation module 64 comprises a database of background scenes. In this case, the simulation generation module 64 is adapted to retrieve a particular scene from the database and insert the virtual representation of the hand within the particular scene.

In another embodiment, the background scene is provided to the simulation generation module 64 by the user or another machine connected to the simulation generation module 64, as described below.

Once the simulation has been generated, the simulation generation module 64 transmits the simulation to the display module 66 so that the user may see the virtual representation of his hand in substantially real-time. It should be understood that the position of the hand within the 3D space is transmitted to the simulation generation module 64 substantially continuously and the simulation generation module 64 generates the simulation in substantially real-time so that any change in the position of the hand within the 3D space is reflected in the position of the virtual representation of the hand within the background scene in substantially real-time.

The simulation generation module 64 is also adapted to transmit the simulation to the activity-specific data generation module 68. In one embodiment, the activity-specific data generation module 68 is adapted to determine the above described activity-specific data from the simulation and/or the position data outputted by the motion tracking module 62. For example, the activity-specific data generation module 68 extracts all information required for generating the activity-specific data such as the position of the virtual representation of the hand, the position of elements of the background scene, the time required to perform a particular task, etc., and determines the activity-specific data using the extracted information.

In another embodiment, the activity-specific data generation module 68 is adapted to receive the position of the hand within the 3D space from the motion tracking module 62 or the simulation generation module 64 instead of or in addition to receiving the simulation form the simulation generation module 64. In this case, the activity-specific data generation module 68 is adapted to use the position of the hand within the 3D space instead of that of the virtual representation of the hand within the background scene for determining the activity-specific data.

In one embodiment, the simulation generation module 64 is further adapted to provide the user with instructions for executing the rehabilitation exercise. For example, the instructions may be visual or written instructions provided to the user via the display module 66 either before the beginning of the simulation or during the simulation. The system 60 may further comprise a speaker connected to the simulation generation module 64, which is then further adapted to provide the user with oral instructions either before the start of the simulation or during the simulation.

In one embodiment, the activity-specific data for different rehabilitation exercises are stored on a local or external memory. The activity-specific data may also be displayed to the user who may see his progression with respect to the results obtained in a previous rehabilitation session.

In one embodiment, the user machine 40 or the simulation generation module 64 is connected to a server via an Internet connection for example, and the background scene and the instructions are received from the server. It should be understood that user may have to identify in order to connect to the server and receive the background scene and the simulation. It should also be understood that some elements of the background scene may be stored on the machine 40 or the simulation generation module 64 while other elements of the background scene are received from the server.

While they may be stored on a local memory, the generated activity-specific data may be sent to the server for being stored thereon. A medical professional in charge of the rehabilitation of the user/patient may then connect to the server via a secured connection to consult the activity-specific data. It should be understood that the medical professional may have to identify himself in order to have access to the activity-specific data of a patient. The server may be adapted to generate graphs, tables, and the like for presenting the activity-specific data to the medical professional. Alternatively, the graphs, tables, etc., may be generated by the machine 40 or the activity-specific data generation module 68 and subsequently sent to the server.

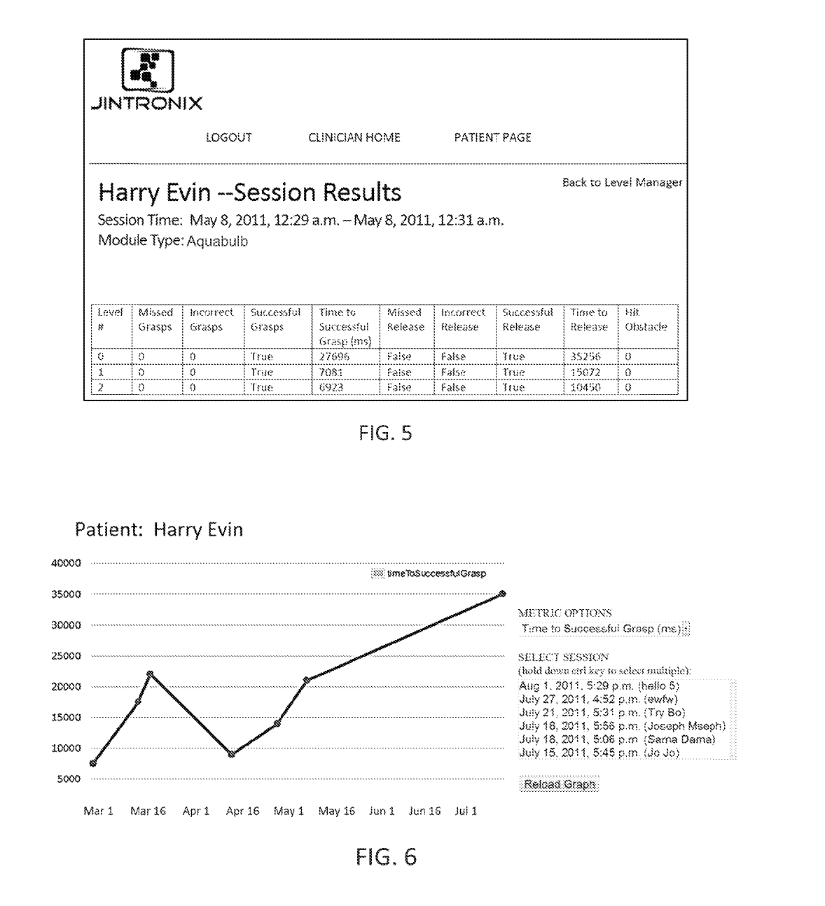

For example, FIG. 5 illustrates a table presenting activity-specific data. The table provides the medical professional with the number of missed grasps, the number of incorrect grasps, the number of successful grasps, the time taken by the patient to execute a successful grasp, etc.

The server may also store the activity-specific data for different rehabilitation exercises executed over time for a same patient and generate a graph presenting the evolution in time of a particular activity-specific data. For example, FIG. 6 illustrates the time taken by a particular patient to execute a successful grasp over time. Using this graph, a medical professional may evaluate the progression of the patient.

The simulation may also be uploaded to the server so that the medical professional may replay the simulation by either playing a pre-encoded video of the simulation, or by re-rendering activity data points using in-browser 3D rendering techniques such as WebGL™ for example. It should be understood that the simulation may be re-rendered on the server and subsequently sent to the medical professional computer, or re-rendered on the medical professional computer.

In the case of playing a pre-encoded video of the simulation, the frames of the interactive scene are recorded in substantially real-time in video format on the user machine as the user engages with the interactive environment and executes the movements.

In one embodiment, as the user engages in the activity, the video is sent substantially in real-time to the server to be stored. In another embodiment, the video is sent to the server after the user engages in the activity.

In one embodiment, the clinician can view the stored video via a web user interface.

In the case of re-rendering activity data points using in-browser 3D rendering techniques such as WebGL™, all patient movement data-points, or certain key patient movement data-points are recorded in real-time by the user machine as the patient engages in the activity.

In one embodiment, as the patient engages in the activity, the points representing the positions of the patients limbs through time (i.e. patient movements) are immediately sent to the server in real-time. Then, by means of in-browser 3D rendering techniques such as WebGL™, the clinician may view a re-rendered playback of the patient's movements.

In one embodiment, when the patient has completed an activity, the points representing the positions of the patient's limbs through time (i.e. patient movements) are immediately sent to the server in real-time. Then, by means of an in-browser 3D rendering techniques such as WebGL™, the clinician may view a re-rendered playback of the patient's movements.

In one embodiment, the server is adapted to inform the medical professional when activity-specific data have been received from a patient. For example, the server may be adapted to send an email to the medical professional in order to inform the medical professional that a particular patient has uploaded activity-specific data.

In one embodiment, the medical professional may also access the server for creating rehabilitation exercises. In this case, the medical professional may generate the background scene to be provided to the patient. In one embodiment, the server comprises a database of different background scenes each corresponding to a corresponding rehabilitation exercise, and the medical professional selects a particular background scene for the patient. Instructions may be associated with each background scene.

In one embodiment, medical professionals have the ability to customize the details of an activity and/or exercise for their patient to perform, by allowing them to choose the background scene, as well as elements that determine the body part required for movement, the type of movement required, and the movement characteristics required.

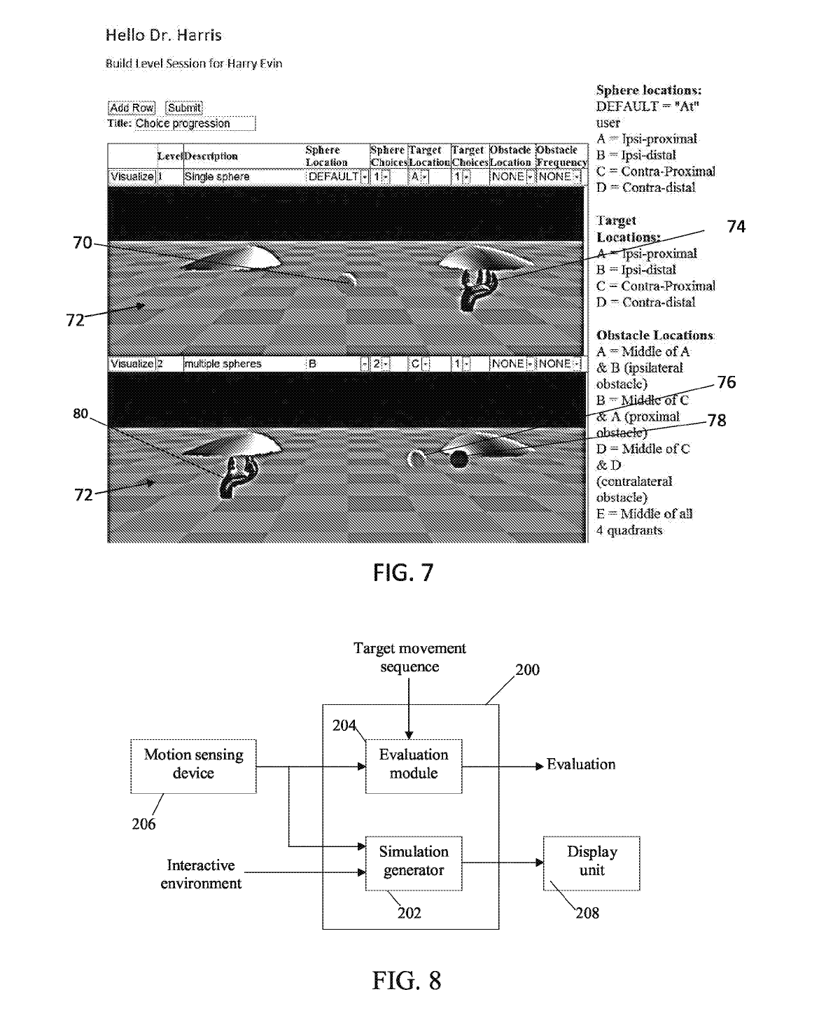

In another embodiment, the medical professional may create the background scene by inserting virtual elements within a background image, as illustrated in FIG. 7. The medical professional is presented with a background image and selects the number, the location, the size, and/or the like, of elements to be inserted in the background image to form the background scene. In the first background scene illustrated in FIG. 7, the medical professional positions a ball 70 to be controlled by the patient movement at a desired initial position within a background image 72 and a receiving hand 74 into which the virtual ball 70 is to be released at a desired target position. In the second background scene illustrated in FIG. 7, two balls 76 and 78 are inserted at desired respective initial positions within the background image 72 and a receiving hand 80 into which the virtual balls 76 and 78 are to be released is positioned at a desired target position.

in one embodiment, the medical professional may precisely position objects which define the start of the required movement, the end of the required movement, and required movement trajectory, such as the balls and receiving hand, by clicking on them and dragging them to a desired location within the background image. In the same or another embodiment, the medical professional chooses between predetermined positions for each element to be positioned within the background image such as the balls 70, 76, 78 and the receiving hands 74 and 80. For example, the predetermined positions may comprise four positions, e.g. the ipsi-proximal, ipsi-distal, contra-proximal, and contra-distal positions.

In one embodiment, an obstacle having the form of a wall may also be inserted in the background image between the ball(s) and the receiving hand for example. The obstacle may have a fixed position or may move within the background image. For example, the obstacle may move upwardly and downwardly at a given frequency. In the last case, the medical professional may set the desired frequency of movement for the obstacle. The medical professional may also set the movement amplitude for the obstacle for example.

In one embodiment, the medical professional may select a body part to be exercised by the patient, e.g. right arm, left arm, left shoulder, trunk, wrist, etc.

In one embodiment, the medical professional may define a type of hand/arm movement to be executed by the patient. For example, the medical professional may insert a path to be followed in the background image.

In another embodiment, the medical professional may customize the exact position of some or all the objects which determine the type of movement required, as described above, in order to define the movement required for the patient to execute.

In one embodiment, the medical professional may define the distance of elements of the background scene from center, i.e. the range of motion required to complete task.

In one embodiment, the medical professional may also enter pacing information such as the time required to complete a particular task.

In one embodiment, the medical professional may set the size, shape, color, etc., of the elements inserted in the background image. For example, the medical professional may set the size of the virtual balls 70, 76, and 78, and the size of the receiving hands 74 and 80.

In one embodiment, the medical professional may set the behavior of the elements inserted in the background image to form the background scene. For example, the clinician may set the speed of the objects, movement trajectory of the objects, or movement path of the objects. As another example, the medical professional may set how the objects shall respond when the user interacts with them.

When such settings are configured by the medical professional, the resulting settings data are written to a storage location on the server. These settings data are instructions that the user machine executes, as described below. These instructions may define aspects such as the positions of the objects (for example: 3D, 2D, or 1D coordinates), characteristics (for example: color, shape, size, etc.), or behavior (for example: movement speed, movement trajectory, movement path, etc.).

In one embodiment, the medical professional may set handicap settings. For example, at a certain handicap setting, only a partial grasp may be required in order to succeed, whereas for the same activity at another handicap setting, a full grasp may be required in order to succeed.

In one embodiment, the user connects to the server and downloads the background scene corresponding to the created rehabilitation exercise, and the corresponding instructions, if any. In one embodiment, the entire background scene including the background image is downloaded by the user. In another embodiment, only some relevant information such as the initial position of some elements of the background scene is sent to the patient machine which re-renders the background scene using the downloaded information in addition to information already stored thereon such as the background image for example.

In one embodiment, instructions are downloaded to the user machine from the server. The user machine then renders the background scene, as well as the positions, characteristics, and behaviors of the objects according to the instructions it has received.

While the present description refers to a server for presenting activity-specific data to the medical professional, it should be understood that the medical professional computer may receive directly the activity-specific data from the user machine and generate graphs, tables, etc. Similarly, the background scenes may be directly generated on the medical professional computer and directly sent to the user computer.

Referring back to FIGS. 3 and 4, it should be understood that any adequate motion tracking unit, module, or system adapted to track the position of a hand may be used. As previously described, the motion tracking unit is adapted to determine at least the position of the user hand within the 3D space. The motion tracking unit may also be adapted to determine the orientation of the user hand within the 3D space. Furthermore, the motion tracking unit may also be adapted to determine the position of at least one finger of the hand with respect to the hand palm.

In one embodiment, the motion tracking unit comprises an infrared 3D triangulation unit which comprises an infrared (IR) light source secured to the user hand. The emitted IR light is captured by two cameras mounted side-by-side. Standard computer-vision methods are employed to detect the horizontal and vertical coordinates of the IR light source, and stereo parallax methods are employed to determine the depth of the IR light source.

In another embodiment, the motion tracking unit comprises a contour detection unit. Color-detection and contour-detection algorithms are employed over at least two cameras mounted side-by-side in order to separate the foreground from the background, and identify the position of the user's hand. Stereo parallax methods are employed to determine the depth of the user's hand.

In still a further embodiment, the motion tracking unit comprises a magnetic tracking system which calculates the position and orientation of the hand using the relative magnetic flux of three orthogonal cons on both the transmitter and each receiver. The relative intensity of the voltage or current of the three coils allows the system to calculate both range and orientation by mapping the tracking volume.

In an embodiment in which the flexion/extension state of the fingers is required, the motion tracking unit may comprise a glove to be worn by the user and having accelerometers, gyroscopes, and variable flexion resistors secured thereto. The accelerometers are used to measure the tilt of the hand and optical fibers are used to measure the angle of bend of the various joints for each finger. The optical fibers are optically coupled to a light source and a light detector. By bending the fiber, some light propagating in the fiber core is coupled into the fiber cladding, and therefore lost. The finger bend can then be determined from the amount of light propagating up to the light detector. For example, the glove may comprise a main circuit integrated with an accelerometer, a second accelerometer, six bend sensors on the joints of the finger; four wrist bend sensors, and finger abduction/adduction bend sensors.

In one embodiment, the above described method and system allow clinicians to design rehabilitation exercises that may be fully customized for patients and allow for a more automated personalized treatment program, and more relevant clinical outcome measures.

In one embodiment, the above described methods and systems promote a greater and more frequent interaction between rehabilitation professionals and patients, while decreasing the resource burden on health-care infrastructure.

In one embodiment, the above described method and system are adapted for upper limb rehabilitation. In this case, abilities such as range of motion, control of movement, balance, speed, accuracy, strength, and/or the like may be assessed. In one embodiment, the method and system are adapted to the rehabilitation of a hand. Functions such as grasp, pinch, rotation, finger tap may be evaluated by providing the patient with activities such as holding items, writing, typing, rotating a door handle, pouring liquids, etc. In another embodiment, the method and system are adapted to the rehabilitation of a forearm (from wrist joint to elbow) and/or an upper arm (elbow to shoulder). Functions such as rotation and/or lateral/vertical/horizontal movement may be evaluated by providing the patient with adequate activities such as moving objects on table, reaching above and below upper body, putting on cloths, driving, and/or the like. In a further embodiment, the method and system are adapted to the rehabilitation of trunk and/or a shoulder. In this case, functions such as lean forward, backward, sideways, and/or rotation may be evaluated by providing the patient with adequate activities such as putting on pants, walking up the stairs, sitting at desk, and/or the like.

In another embodiment, the above described method and system are adapted for lower limb rehabilitation. In this case, abilities such as range of motion, control, balance, strength, and/or the like may be assessed. In one embodiment, the method and system are adapted to the rehabilitation of a foot (from ankle to tip of toes). In this case, functions such as raise feet when walking, rotation, and/or the like may be evaluated by providing the patient with adequate activities such as walking for example. In one embodiment, the method and system are adapted to the rehabilitation of a lower leg (from ankle to knee) and/or an upper leg (including knee and thigh). In this case, functions such as extension, flexion, rotation, and/or the like may be evaluated by providing the patient with adequate activities such as walking, sitting, standing, going up the stairs, bending down to reach for object, putting on pants, dancing, and/or the like. It should be understood that subgroups of muscles in each of the above body parts may be monitored as well.

FIG. 8 illustrates one embodiment of a system 200 for evaluating a patient/user during a virtual-reality rehabilitation exercise. The system 200 comprises a simulation generator 202 and an evaluation module 204. The system 200 is in communication with a motion sensing device 206 and a display unit 208. The system is further in communication with a medical professional machine (not shown) or a server (not shown) to which the medical professional is connected via his medical professional machine.

The system 200 is adapted to generate an interactive simulation to be displayed on the display unit 208. The interactive simulation is generated using an interactive environment received from the medical professional machine or the server. The interactive environment comprises all information required by the simulation generator 202 for generating the interactive simulation. While the simulation is displayed on the display unit 208, the user executes a rehabilitation exercise. The movement(s) of the user during the rehabilitation exercise is(are) tracked by the motion sensing device 206. A visual characteristic of a virtual user-controlled object, such as the position of the virtual user-controlled object, comprised in the simulation is modified as a function of the movement of the user, thereby rendering the simulation interactive.

The evaluation module 204 receives a target movement to be executed during the rehabilitation exercise from the medical professional machine or the server. In one embodiment, the target movement comprises a sequence of at least two target elementary movements to be executed by the user. The evaluation module 204 compares the movement executed during the rehabilitation exercise and tracked by the motion sensing device 206 to the target movement. In one embodiment, the evaluation module 204 breaks the executed movement captured by the motion sensing device 206 into elementary movements which are each compared to a respective target elementary movement.

Using the results of the comparison, the evaluation module 204 outputs an evaluation of the rehabilitation exercise performed by the user, which indicates whether the user has successfully executed the movement or whether the user failed. The evaluation may be stored locally on a storing unit and/or transmitted to the server of the medical professional machine, for example.

First, a medical professional determines which movement should be performed by the user as a function of the medical needs for the user, i.e. a target movement. In one embodiment, the target movement comprises a sequence of at least two target elementary movements. An elementary movement is defined as a body movement type for a given body part of the user according to a single degree of freedom. In the sequence, each elementary movement may also be characterized by a respective range of movement. Examples of body movement types comprise flexion, extension, adduction, abduction, flexion, rotation, pronation, supination, etc. Examples of body parts comprise a trunk, a joint such as left knee, right shoulder, left elbow, left wrist, an ankle, and/or the like, etc.

It should be understood that a sequence of target movements comprises at least two different elementary movements and/or at least two different movement ranges, i.e. the body part and/or the type of movement and/or the range of movement for the at least two different elementary movements is different. For example, the body parts for the two elementary movements may be different while the movement types and the ranges of movement may be substantially identical. In another example, the movement types for two elementary movements may be different while the body parts and the ranges of movement may be substantially identical. In a further example, the ranges of movement for two elementary movements may be different while the body parts and the types of movement may be identical. In another example in which a sequence comprises three elementary movements, two of the three elementary movements may be substantially identical as long they are not performed concurrently and they are different from the third elementary movement.

It should also be understood that a sequence of elementary movement may comprise at least two elementary movements which are temporally ordered, i.e. each elementary movements is assigned a temporal position indicating the time at which it is to be executed. In one embodiment, the at least two elementary movements to be executed substantially concurrently. Alternatively, the at least two elementary movements may be executed sequentially. For example, a sequence may comprise a first elementary movement and a second elementary movement to be executed substantially concurrently, and a third elementary movement to be performed subsequently to the execution of the first two elementary movements.

In one embodiment, a target elementary movement may further be characterized by additional properties such as coordination characteristics, endurance characteristics, and/or cognition characteristics. In this case, each target elementary movement is further characterized by at least one additional property and a corresponding target value or target range of value for the additional property.

Examples of adequate coordination characteristics comprise a movement speed, a movement precision, a balance, a movement compensation, a bilateral coordination, etc. The movement speed is defined as a distance achieved during an elementary movement over the time period taken for achieving the elementary movement. The movement precision represents the patient's deviation from a target movement axis during the execution of the elementary movement. A movement precision may be expressed as an angle, or a unit of distance. A movement precision may be determined by measuring an average angle or path deviance from a targeted axis, measuring the maximum angle or path deviance from a targeted axis, measuring the time spent outside an acceptable precision range, etc. The balance is defined as the level of body stability as an elementary movement is executed, and may be determined by measuring the position of the trunk and detecting the level of erratic movement of the trunk. The movement compensation is defined as the position/angle of the trunk or shoulders as a movement is executed, and indicated how much trunk movement was used to aid in the execution of a particular task. The movement compensation may be determined by calculating the value of the maximum or average trunk angle as a patient engages in an elementary movement. The bilateral coordination is defined as the level of independence between body-parts of the patient's separate limbs. For example, the bilateral coordination may be determined by measuring the level of successful precision of a body-part on one limb as a function of independent movement complexity of the body-parts on the limb of the opposite side, calculating the level of success of a task specific movement involving the patient's two arms, etc.

Examples of adequate endurance characteristics comprise the movement consistency over time, compensation patterns over time, and the like. The movement consistency over time can be calculated when elementary movements are repeated over an activity, and is defined as the amount of movement deviation increase over repetitions. The movement consistency over time may be determined by comparing the precision value of a movement of the first movement repetition (or first set of repetitions) with the precision value from the last movement repetition (or last set of repetitions). Based on this, the percentage of increase of movement deviation can be calculated. The compensation patterns over time can be calculated when elementary movements are repeated over an activity, and is defined as the amount of increase of trunk or shoulder compensation over repetitions. The compensation patterns over time may be determined by comparing the amount of compensation (i.e. posture) from the first repetition (or first net of repetitions) to the amount of compensation (i.e. posture) from the last repetition (or last set of repetitions).

Examples of cognition characteristics comprise the memory, the attention, the pattern recognition, the sequence cognition, the reaction, the choice reaction, the executive functioning, etc. The memory is defined as the level of retention of information. One method to calculate level of memory is to present information to the patient, and then hide it, and then after a period of time, present a choice that requires the recall of the original information. Success can be measured as a function of whether the correct choice was made as a function of time elapsed.

After creating the sequence of the target movements, the medical professional creates an interactive environment which comprises all necessary information for the simulation generator 202 to generate the interactive simulation. The interactive simulation provides the user with the interactive environment in which the user has to execute a task. The execution of the task requires that the user performs the sequence of elementary movements. The interactive environment comprises at least a virtual user-controlled element or object, i.e. a virtual element of which at least one characteristic is to be controlled by the movement of the user during the simulation and the execution of the rehabilitation exercise.

In one embodiment, the interactive environment is defined only by a virtual user-controlled element of which at least one characteristic is to be controlled by the movement of the user. Examples of adequate characteristic to be controlled comprise the position, the size, the color, the shape, or the like. The interactive environment further comprises an identification of the target movement.

In one example, the interactive environment comprises a ball of which the size is to be controlled by the movement of the user during the simulation. The task to be executed by the user consists in increasing the size of the virtual ball until it explodes. The corresponding sequence of elementary movements is presented in Table 1. The first step of the sequence consists in getting into position: straightening the elbow, and moving the arm to the left side. The second step consists in moving the arm along the straight horizontal line to the right.

TABLE 1
Sequence of elementary movements for moving
a hand along a horizontal line.

| | Simple Movement | Simple Movement | Simple Movement |
|---|---|---|---|
| Step | Tasks (A) | Tasks (B) | Tasks (C) |
| 1 | Shoulder Forward | Shoulder Horizontal | Elbow Extension 90′-0′ |
| | Flexion 0′-90′ | Adduction 90′-120′ | |
| 2 | | Shoulder Horizontal | Elbow Stasis |
| | | Abduction 120′-60′ | |

The user is instructed to move his hand along an imaginary horizontal line during the simulation in order to increase the size of the ball. As the user moves his hand along the imaginary horizontal line, the size of the ball increases and explodes to indicate that the user has completed the target movement. In this case, the interactive environment further comprises the given movement allowing the execution of the task, i.e. the imaginary horizontal line to be followed by the user during the simulation. It should be understood that the given movement allowing the execution of the task is determined as function of the corresponding sequence of elementary movements and the execution of this given movement requires the execution of the sequence of elementary movements.

In another embodiment, the interactive environment comprises at least one virtual user-controlled element and at least one virtual reference element. At least one characteristic of the at least one virtual reference element is chosen as a function of the target movement to be executed by the user during the rehabilitation exercise. In one embodiment, the position of the at least one virtual reference element is chosen as a function of the target movement to be executed by the user during the rehabilitation exercise. Characteristics such as a dimension and/or an orientation may also be determined as a function of the target movement to be executed by the user during the rehabilitation exercise. When the interactive environment comprises at least two virtual elements, the distance between the virtual reference elements may also be determined as a function of the target movement.

In one example, the virtual reference object may be a horizontal line segment and the virtual user controlled element may be a ball. During the simulation, the user has to execute a task, i.e. moving the ball along the line. The corresponding sequence of elementary movements is presented in Table 1. The user is instructed to move his hand in order to move the ball. In this case, the position of the center of the line segment and the orientation and length of the line segment are chosen as a function of the movement to be executed during the rehabilitation exercise.

In another example, the interactive environment comprises five food objects and the virtual user-controlled element may be a fish. During the simulation, the task of the user is to move the fish so that it eats the five pieces of food. The user is instructed to move his right hand to control the fish. The goal of this rehabilitation exercise to have the user executing the same sequence of elementary movements as that in the previous example. In this case, the position of the pieces of food is chosen as a function of the sequence of elementary movements. For example, the five pieces of food may be aligned along a horizontal line. In this case, the interactive movement comprises the position for the five food object in addition to characteristics such as their shape, their color, their dimensions or size, and/or the like. The interactive environment further comprises characteristics for the fish such as its shape, color, dimensions, and/or the like.

In one embodiment, the position of the virtual reference element is fixed and may not change during the interactive simulation. In another embodiment, the position of the virtual reference element may change during the simulation. For example, the virtual reference element may be ball moving from top to bottom and the user may be asked to catch the falling ball. In this case, the interactive comprises all information for animating the moving object such as a speed, an initial position, a final position, and/or the like.

In one embodiment, the difficulty of a rehabilitation exercise may be controlled by at least one characteristic of the virtual reference element and/or the virtual user-controlled element. Referring back to the example in which the user has to move his hand so that a ball moves along a horizontal line segment, the difficulty of the rehabilitation exercise may be set by the thickness of the line segment. Increasing the thickness of the line segment decreases the difficulty for the user to move the ball along the line segment.

In one embodiment, the medical professional machine or computer is adapted to allow the user to input the target movement and the interactive environment. In another embodiment, the medical professional connects to server via a medical professional machine and the server is adapted to allow the user to input the target movement and the interactive environment.

In one embodiment, the medical professional machine or the server comprises a database of elementary movements. The medical professional may then create the target movement for the user by selecting at least two different elementary movements from the database and defining a temporal order of execution for elementary movements. In one embodiment, the database further a predetermined target range of movement for each elementary movement stored therein. It should be understood that the medical professional may modify the ranges of movement to reflect the particular needs of the user. In another embodiment, the database comprises no predetermined ranges of movement for the elementary movements. The medical professional is then requested to input a respective range of movement for each selected elementary movement.

In one embodiment, the database comprises at least one target functional movement or functional task such as brushing teeth, hanging a coat, cooking, self-grooming, and/or the like. For each functional task, the database comprises a corresponding sequence of target elementary movements. Therefore, the medical professional may select a desired functional task from the database. The medical professional machine or the server retrieves the sequence of target elementary movements corresponding to the selected functional task and transmits the retrieved sequence to the evaluation module 204. Table 2 illustrates a sequence of target elementary movements corresponding the “hanging a coat” functional task.

TABLE 2
Sequence of target elementary movements contained
in the “hanging a coat” functional task.

| Steps | Shoulder | Elbow | Wrist | Fingers/Thumb |
|---|---|---|---|---|
| 1 | Shoulder Abduction 0′-15′ | | Wrist abduction 0′-20′ | grip (coat |
| | | | | hanger) |
| 2 | Shoulder Flexion 0′-45′ | Elbow Flexion 0′-90′ | | static grip |
| 3 | Shoulder Flexion 45′-90′ | Elbow Extension 90′-0′ | | |
| 4 | Shoulder Flexion 90′-120′ | Forearm Rotation | | |
| | | 90′-100′ | | |
| 5 | Shoulder Stasis - | | Wrist adduction | Release Grip |
| | Active | | 20′-(-)20′ | |
| 6 | Shoulder Extension | | Wrist abduction (-) | |
| | 120′-90′ | | 20′-0′ | |
| 7 | Shoulder Extension | | | |
| | 90′-45′ | | | |
| 8 | Shoulder Extension | | | |
| | 45′-0′ | | | |

It should be understood that the database may alternatively be stored in the evaluation module 204. In one embodiment, the database comprises predetermined sequences of elementary movements. In this case, the evaluation module 204 may receive an identification for a desired sequence from the medical professional machine or the server, and retrieves the corresponding desired sequence from the database. In another embodiment, the database may comprise predetermined elementary movements and, optionally, corresponding movement ranges. In this case, the evaluation module 204 receives an identification for at least two elementary movements from the medical professional machine or the server, and retrieves the corresponding elementary movements. If a movement range is associated with each elementary movement in the database, the evaluation module 204 further retrieves the corresponding movement range for each retrieved elementary movement. In an embodiment, in which the database comprises no movement ranges, then the evaluation module 204 receives the movement ranges along with the identification of the elementary movements.

The database may further comprise a corresponding interactive environment for each possible sequence of target movements. In this case, the server or medical professional machine may automatically retrieve the interactive environment corresponding to the sequence of target elementary movements generated by the medical professional and transmits the retrieved interactive environment to the simulation generator 202 in addition to transmit the sequence of target elementary movements to the evaluation module 204. In one embodiment, the database may comprise at least two corresponding interactive environments for at least one given sequence of the target elementary movements. In this case, the user chooses between the different interactive environments to select a desired one. The selected interactive environment is then sent to the simulation generator 202.

In one embodiment, the interactive environment is created in two steps: the selection of a scenario and the customization of the scenario. A scenario is an incomplete interactive environment, and comprises at least one virtual user-controlled element and at least one virtual reference element of which the characteristics are not specified. The server or medical professional machine may provide the medical professional with at least one scenario that corresponds to the sequence of target elementary movements inputted by the medical professional. Alternatively, the server or medical professional machine may provide the medical professional with all of the scenarios and the medical professional has to determine a particular scenario that corresponds to the sequence of target elementary movements that he inputted. After selecting a desired scenario, the medical professional customizes the scenario by specifying the characteristics of the virtual reference elements.

It should be understood that a database for interactive scenarios may be stored in the simulation generator 202. In one embodiment, the database comprises a set of complete interactive environments. The simulation generator 202 then receives an identification of a desired interactive scenario from the medical professional machine or the server, and retrieves the desired interactive scenario from the database. In another embodiment, the database may comprise incomplete interactive scenarios. For example, an incomplete interactive scenario may be an interactive scenario having missing information. For example, the position of the virtual elements may be missing. In this case, the simulation generator 202 receives an identification of a desired incomplete scenario and the missing information from the medial professional machine or the server, retrieves the incomplete scenario from the database, and adds the received missing information to the retrieved incomplete scenario to complete the interactive scenario.

In an example, a rehabilitation exercise focuses on unilateral shoulder and elbow movements with a focus on constant movement accuracy. The interactive environment comprises a fish whose 3D position is controlled by the user's shoulder and elbow movements and virtual reference elements. The virtual reference elements comprise a sequence of food objects that determine the path to guide the user during the simulation, a piranha that may chase the fish during the simulation, and moving obstacles for the fish. The interactive environment may further comprise a 3D underwater background scene. During the simulation, the user is requested to move his hand to control the fish so that the fish eat the food objects. The user has to move the fish at a minimal speed in order to escape the piranha.

In accordance with the medical needs of the user, the medical professional customizes the scenario by specifying the position of the food objects to provide a shape to the sequence of food objects. For example, the food objects can be arranged in the shape of a square, a triangle, a figure eight, etc. It should be understood that the shape of the sequence of food objects is chosen as a function of the target elementary movements. The medical professional further specifies the size of the food objects to set the required precision of user's movement path, the speed of the piranha to set the minimum speed required by the patient's movement, the frequency and/or position of the obstacles, and the number of movement repetitions required.

In one embodiment, the medical professional machine or the server displays to the medical professional an interface comprising the 3D background scene in which the obstacles and the food objects are inserted. The medical professional may then click and drag the obstacles and the food objects at a desired place within the 3D background scene to specify their position.

It should be understood that an interactive environment comprises all necessary information to generate a 2D or 3D interactive simulation. Similarly, the simulation generator 202 is adapted to generate a 2D or a 3D interactive simulation.

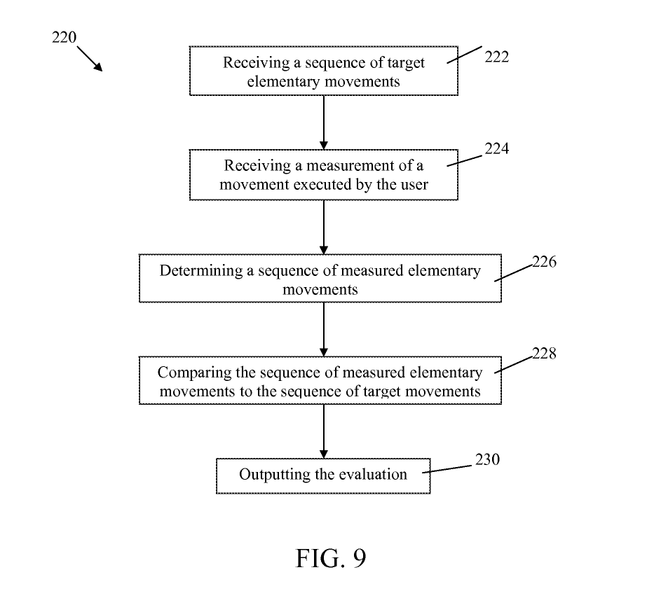

Referring to FIGS. 8 and 9, the evaluation module 204 receives the sequence of target elementary movements and their respective target range of movement from the medical professional machine or the server at step 222. The simulation generator 202 receives the interactive environment and generates a simulation, which is sent to the display unit 208. The motion sensing device 206 tracks the movements executed by the user and the simulation generator updates the characteristic(s) of the virtual user-controlled element within the interactive simulation according to the received measured movement received from the motion sensing device 206. The interactive simulation provides a virtual reality environment in which the user executes a task.

The motion sensing unit 206 measures the movement of the user during the rehabilitation exercise and transmits the measured movement executed by the user to the evaluation unit 204, at step 224.

In one embodiment, the characteristic of the user-controlled element is controlled by the movements of the body parts defined in the sequence of target elementary movements. In this case, the motion sensing device 206 transmits the measured elementary movements of the body parts and their respective movement range to the simulation generator 202 which modifies, in substantially real-time, the characteristic of the virtual user-controlled element within the simulation according of the received measured movements of the body parts.

In another embodiment, the characteristic of the user-controlled element is controlled by the movement of a reference point of the user body, of which the position changes during the execution of the sequence of target elementary movements by the user. In this case, the motion sensing device 206 is further adapted to track the position of the reference point and transmits the measured 3D position of the reference point to the simulation generator 202 which modifies, in substantially real-time, the characteristic of the virtual user-controlled element within the simulation according of the received measured movements of the body parts. For example, the sequence of target elementary movements may include movements of the elbow and the shoulder while the position of the wrist may be sent to the simulation generator 202. Since a movement of the elbow and/or shoulder triggers a change of position of the wrist, the position of the virtual user-controlled element within the simulation is adjusted as a function of the received position of the user wrist. In a further embodiment, the characteristic of the user-controlled element is controlled by the relative position between a body part and a reference point such as the relative position between a wrist and a reference point on the chest of the user.

At step 224, the evaluation module 204 receives the measurement of the movement executed by the user during the simulation from the motion sensing device 206, and determines, from the measurement of the user movement, a sequence of measured elementary movements executed by the user, at step 226.

In one embodiment, the motion sensing device 206 is adapted to measure the elementary movements defined in the sequence of target elementary movements. In this case, the evaluation module 204 receives a measured range of movement in time for each elementary movement defined in the sequence of target elementary movements, and determines the sequence of elementary movements performed by the user by determining the order in which the elementary movements have been executed by the user.

For example, the motion sensing device 206 may comprise a first sensor adapted to only measure elbow flexion, and a second sensor adapted to only measure shoulder adduction. In this case, when it receives a first range of movement from the first sensor, the evaluation module 204 determines that the first range of movement correspond to the elbow flexion type of movement. When it receives a second range of movement from the second sensor, the evaluation module 204 determines that the second range of movement correspond to the shoulder adduction type of movement. Then, the evaluation module 204 determines the execution order for the elbow flexion and the shoulder adduction by comparing the starting times at which the uses executes the elbow flexion and the shoulder adduction, for example, thereby obtaining the sequence of elementary movements performed by the user during the rehabilitation exercise.

In another embodiment, the evaluation module 204 receives position information for the body parts identified in the target sequence, from which the type of elementary movement has first to be determined in order to determine the sequence of elementary movements. In this case, the evaluation module 204 is adapted to determine the number of elementary movements executed by the user, the type of each elementary movement executed by the user, their respective order, and optionally their respective range of movement and/or additional properties.

For example, the motion sensing device 206 may be adapted to measure position information such as angles associated with body parts, 3D, 2D, or 1D positions of reference points or body parts, relative positions of body parts with respect to reference points, and/or the like. In this case, the evaluation module 204 receives the value in time of the position information for the body parts, and determines the sequence of elementary movements executed by the user, i.e. the number of elementary movements and their temporal execution order, and, for each elementary movement, the corresponding type of movement and the range of movement.

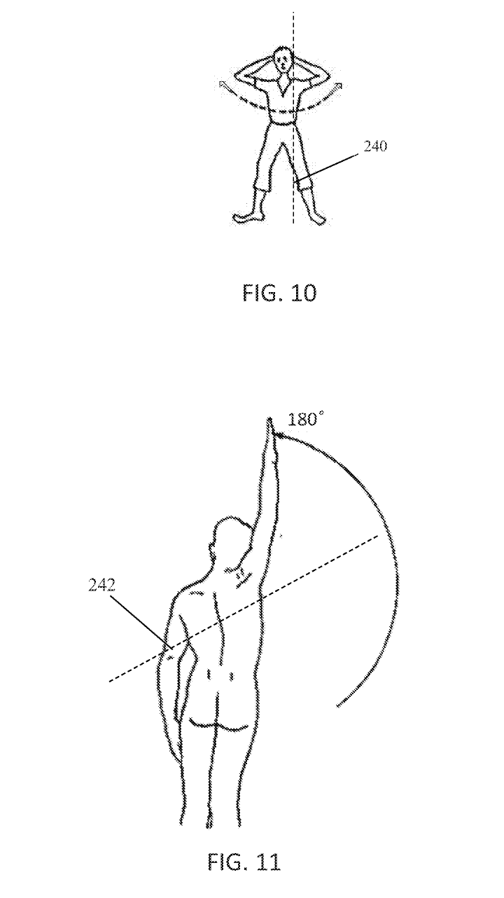

In one embodiment, a type of movement for a given body part is associated with a corresponding movement axis for the given body part, and can therefore be determined by the evaluation module 204 from the identification of the movement axis and the body part. In this case, the evaluation module 204 may comprise a database comprising a respective type of movement for at least some pairs of body part and movement axis. For example, FIG. 10 illustrates a user while rotating his trunk. The motion sensing device 206 may be adapted to measure the 3D position in time of the right shoulder, the left shoulder, and the pelvis of the user. This position information is sent to the evaluation module 204. From the position in time of the shoulders and the pelvis, the evaluation module 204 determines that the trunk of the user rotates about an axis 240 which is comprised in the sagittal plane of the user body and intersects the pelvis and the middle point of the segment extending between the two shoulders. Since, for a trunk, the rotation about the axis 240 is associated with a rotation, the evaluation unit 204 determines that the type of movement exerted by the user is a trunk rotation.

From the value in time of the 3D position of the shoulders and the pelvis, the evaluation module 204 then determines the range(s) of movement. For example, the evaluation module 204 may determine a first rotation from 0′ to 30′ and a second rotation from 30′ to −20′. The evaluation module 204 then determines that two trunk rotations have been executed by the user, i.e. a first trunk rotation from 0′ to 30′ and a second rotation from 30′ to −20′ executed after the first trunk rotation. The evaluation module 204 has then determined, from the position information, the number of elementary movements and their temporal execution order, and, for each elementary movement, the corresponding type of movement and the range of movement, thereby obtaining the sequence of measured elementary movements and their respective range of movement.

In another embodiment, a type of movement for a given body part is associated with a corresponding movement axis for the given body part and a movement direction, and can therefore be determined by the evaluation module 204 from the movement direction and the identification of the movement axis and the body part. In this case, the database of the evaluation module 204 may comprise at least one type of movement to which a respective body part, a respective movement axis, and a respective movement direction are associated.

For example, FIG. 11 illustrates a user while raising an arm from 0′ to 180′. The evaluation module 204 receives the value in time for the shoulder angle, i.e. the angle between the arm and the trunk of the user, from the motion sensing device 206. Since the shoulder angle is associated with a rotation of the arm about an axis 242 which is orthogonal to the coronal plane and intersects the shoulder of the user, the evaluation unit determines that the user rotated his arm about the axis 242. The evaluation module 204 further determines that the shoulder angle increased during the movement. In the database, the combination of the axis 242 (or angle shoulder), an increase of the angle, and the shoulder is associated with a shoulder abduction. Therefore, the evaluation module 204 determines that the user performed a shoulder abduction from 0′ to 180′.

In one embodiment, the motion sensing device 206 is adapted to output the position in time of adequate reference points and the evaluation unit 204 is adapted determine the measured range of movement for the position in time of the reference points. For example, the motion sensing device 206 may be adapted to track and output the position in time of the wrist, elbow, and shoulder of the user. The evaluation unit 204 is then adapted to determine the variation of the angle formed by the arm and the forearm of the user using the position in time of the wrist, elbow and shoulder of the user.

At step 228, the evaluation module 204 compares the sequence of measured elementary movements to the sequence of target elementary movements. Each measured elementary movement is compared to its respective target elementary movement. If the type of movement and the body part associated with the given measured elementary movement respectively matches the type of movement and the body part associated with its respective target elementary movement (i.e. the target elementary movement occupying the same position in the target sequence than the position of the given measured elementary movement in the measured sequence), then the evaluation module 204 determines that the user successfully executed the given target movement. The results of the comparison correspond the evaluation of the performance for the rehabilitation exercise, which is outputted at step 230. The evaluation may be stored locally or remotely in a storing unit. The evaluation may also be sent to the medical professional machine or the server, for example.

in an embodiment in which the target sequence of movement further comprises a range of movement and/or additional properties such as coordination characteristics, endurance characteristics, and/or cognition characteristics for at least one given target elementary movement, the evaluation module 206 is further adapted to determine the range of movement and/or the additional properties for the measured elementary movement corresponding to the given target elementary movement, as described above, and compare the determined value of the range of movement and/or the determined value for the additional properties to their respective target value. The result of the comparison is then included in the evaluation report. For example, for each measured elementary movement, the evaluation module 204 may determine that the elementary movement was adequately executed by the user if the measured range of movement matches its corresponding target range of movement and if the measured elementary movement correspond to its corresponding target elementary movement. Otherwise, the evaluation module 204 may determine that the user failed to execute the target elementary movement.

In one embodiment, the user may perform an inadequate or erratic movement between the execution of the successive target elementary movements while interacting with the simulation and performing the rehabilitation exercise. In this case, the evaluation module 204 may determine that the user has failed to execute the target sequence. For example, the target sequence may comprise an elbow flexion followed by a shoulder adduction. The valuation module 206 may determine that the user first performed an elbow flexion, then a shoulder internal rotation, a shoulder external rotation, and finally a shoulder adduction. The shoulder internal rotation and the shoulder external rotation then form the erratic movement. The evaluation module 204 compares the sequence of elementary movements executed by the user to the sequence of target elementary movements and determines that the second elementary movement executed by the user, i.e. the shoulder internal rotation, does not match the second target movement, i.e. the shoulder adduction. In this case, the evaluation module 204 determines that the user failed to execute the second target elementary movement.

In another embodiment, the evaluation module 206 may first compare each elementary movement executed by the user to all of the target elementary movements except the target elementary movements that have already been matched with a respective executed elementary movement. For example, the elbow flexion executed by the user is compared to the first target movement and a match between the two is found. The shoulder internal rotation and the shoulder external rotation are then each compare to the other target elementary movements in the sequence, i.e. the shoulder adduction, and no match is found. Finally, the shoulder adduction executed by the user is then compared to the other target elementary movements in the sequence, i.e. the shoulder adduction, and a match is found. The evaluation module 204 then determines that the first executed target movement matches the first target elementary movement, and the fourth executed elementary movement matches the second target elementary movement. In this case, the evaluation may consider the second and third elementary movements, i.e. the shoulder internal rotation and the shoulder external rotation, as being part of an erratic movement and ignore them. The evaluation module 204 than determines that the sequence of elementary movements executed by the user only comprises the first and fourth elementary movements executed by the user, i.e. the elbow flexion and the shoulder adduction, and determine that the user successfully executed the sequence of target elementary movements.

The medical professional may then consult the evaluation of the performance of the user during the rehabilitation exercise. The evaluation provides an identification of the elementary movements that the user successfully executed and the elementary movements that the user failed to adequately execute. The medical professional may then prescribe a rehabilitation exercise that focuses on the elementary movement(s) that the user failed to adequately execute, decrease the difficulty related to the failed elementary movement(s) in the same rehabilitation exercise, etc. Alternatively, if the user successfully executed all of the elementary exercises, the medical professional may create another rehabilitation exercise having a greater difficulty, focusing on other elementary movements, etc.

In an embodiment in which the sequence of target elementary movements comprises a target value or range of value for an additional property characterizing at least one elementary movement, the evaluation module 204 is further adapted to determine the additional property value for the corresponding executed elementary movement and compare the determined value to the target value. The comparison is then part of the evaluation of the user.

In one embodiment, the additional property may be determined from the data received from the motion sensing device 206. In the same or another embodiment, the additional property is determined from the interactive simulation. In this case, interactive simulation data may be sent from the simulation generator 202 to the evaluation module 204.

For example, the evaluation module 204 may be adapted to determine a movement deviation or movement precision. Referring back to the example in which the task of the user consists in moving a ball along a horizontal line segment, simulation data are sent from the simulation generator 202 to the evaluation module 204. The simulation data comprises the position of the horizontal line segment and the position of the ball during the interactive simulation. The evaluation module 204 then determines the movement deviation as being the maximal distance between the ball and the line during the simulation. The movement deviation indicates the capacity of the user to move his hand represented by the ball along a predetermined path represented by the line segment. The evaluation module 204 then compares the determined deviation to a target deviation contained in the received sequence of the target movements. If the determined deviation is below or equal to the target deviation, the evaluation module 204 determined that the user was successful. The result of the comparison is included in the outputted evaluation.

The following presents an example in which the evaluation module 204 receives a sequence of eleven target elementary movements, as illustrated in Table 3, and target coordination and cognition characteristics associated with the shoulder elementary movements and the elbow elementary movements, as illustrated in Tables 4 and 5, respectively. The sequence comprises height successive elementary movements for a shoulder and three successive elementary movements for an elbow. A target range of movement is associated with each elementary movement. The first elbow elementary movement is to be executed substantially concurrently with the second shoulder elementary movement, the second elbow elementary movement is to be executed substantially concurrently with the third shoulder elementary movement, and the third elbow elementary movement is to be executed substantially concurrently with the fourth shoulder elementary movement.

TABLE 3
Sequence of shoulder and elbow elementary movements.

| | Shoulder elementary | Elbow elementary |
|---|---|---|
| Step | movement | movement |
| 1 | Shoulder Abduction 0-15′ | |
| 2 | Shoulder Flexion 0-45′ | Elbow Flexion 0-90′ |
| 3 | Shoulder Flexion 45′-90′ | Elbow Extension 90′-0′ |
| 4 | Shoulder Flexion 90′-120′ | Forearm Rotation 90′-100′ |
| 5 | Shoulder Stasis-Active | |
| 6 | Shoulder Extension 120′-90′ | |
| 7 | Shoulder Extension 90′-45′ | |
| 8 | Shoulder Extension 45′-0′ | |

TABLE 4
Target coordination and cognition characteristics for shoulder
elementary movements.

| | | Coordination | | | | |
|---|---|---|---|---|---|---|
| | Shoulder | | | Posterior | | Cognition |
| | elementary | Movement | | Trunk | Lateral Trunk | Reaction |
| Step | movement | Precision | Speed | Compensation | Compensation | Time |
| 1 | Shoulder | +/−9 cm | 10 cm/sec | 10 cm | 10 cm | 1.5 secs |
| | Abduction | | | | | |
| | 0-15′ | | | | | |
| 2 | Shoulder | +/−9 cm | 10 cm/sec | 10 cm | 10 cm | N/A |
| | Flexion 0-45′ | | | | | |
| 3 | Shoulder | +/−9 cm | 10 cm/sec | 10 cm | 10 cm | N/A |
| | Flexion 45-90′ | | | | | |
| 4 | Shoulder | +/−9 cm | 10 cm/sec | 10 cm | 10 cm | N/A |
| | Flexion 90′-120′ | | | | | |
| 5 | Shoulder | +/−9 cm | 10 cm/sec | 10 cm | 10 cm | N/A |
| | Stasis - | | | | | |
| | Active | | | | | |
| 6 | Shoulder | +/−9 cm | 10 cm/sec | 10 cm | 10 cm | 1.5 secs |
| | Extension | | | | | |
| | 120′-90′ | | | | | |
| 7 | Shoulder | +/−9 cm | 10 cm/sec | 10 cm | 10 cm | N/A |
| | Extension | | | | | |
| | 90′-45′ | | | | | |
| 8 | Shoulder | +/−9 cm | 10 cm/sec | 10 cm | 10 cm | N/A |
| | Extension | | | | | |
| | 45′-0′ | | | | | |

TABLE 5
Target coordination and cognition characteristics for elbow elementary
movements.

| | | Coordination | | | | |
|---|---|---|---|---|---|---|
| | Elbow | | | Posterior | | Cognition |
| | elementary | Movement | | Trunk | Lateral Trunk | Reaction |
| Step | movement | Precision | Speed | Compensation | Compensation | Time |
| 2 | Elbow | +/−9 cm | 10 cm/sec | 10 cm | 10 cm | 1-5 secs |
| | Extension | | | | | |
| | 60′-160′ | | | | | |
| 3 | Elbow | +/−9 cm | 10 cm/sec | 10 cm | 10 cm | N/A |
| | Flexion | | | | | |
| | 160′-60′ | | | | | |
| 4 | Forearm | +/−9 cm | 10 cm/sec | 10 cm | 10 cm | N/A |
| | Rotation 60′-100′ | | | | | |

In this rehabilitation exercise, the user is asked to execute at least one task in the interactive simulation. It should be understood that different interactive environments and simulations may be created to correspond to the present sequence. For example, the user may be requested to move his hand to execute the different tasks and therefore, execute the sequence of elementary movements.

The sequence of elementary movements further comprises target values for coordination characteristics and for a cognition characteristic, for each elementary movement. The coordination characteristics comprise a movement precision which indicates the deviation of the hand of the user form a target path during the simulation, the speed at which the user moves his hand during the execution of an elementary movement, a posterior trunk compensation which is defined as the distance of forward-back movement performed by the trunk to aid in an upper-body movement, and a lateral trunk compensation which is defined as the distance of side-to-side movement made by the trunk to aid in an upper-body movement. The cognition characteristic comprises a reaction time which is defined as the amount of time it took for the patient to perform a correct movement in reaction to a certain reference object provided in the simulation.

The evaluation module 204 receives the measured range of movement for each one of the eleven elementary movements from the motion sensing device 206 in addition to the position of the trunk while the user is executing the elementary movements. The evaluation further calculates the speed of movement of the hand while the user executes each elementary movement, and determines the posterior and lateral trunk compensations using the received position or orientation for the trunk while the user executes each elementary movement. The evaluation module 204 further receives simulation data from the simulation generator 202, and determines the movement precision and reaction time from the received simulation data. Exemplary measured ranges of movements and coordination and cognition characteristics are presented for the shoulder elementary movements and the elbow elementary movements in tables 6 and 7, respectively.

TABLE 6
Measured ranges of movement and coordination and cognition
characteristics for shoulder elementary movements

| | | | Coordination | | | | |
|---|---|---|---|---|---|---|---|
| | | | | | Posterior | Lateral | Cognition |
| | Elementary | Measured | Movement | | Trunk | Trunk | Reaction |
| Step | movement | range | Precision | Speed | Compensation | Compensation | Time |
| 1 | Shoulder | 0-15′ | +/−5 cm | 5 cm/sec | 4 cm | 4 cm | 2.2 secs |
| | Abduction | | | | | | |
| | 0-15′ | | | | | | |
| 2 | Shoulder | 0-45′ | +/−9 cm | 5 cm/sec | 25 cm | 4 cm | N/A |
| | Flexion 0-45′ | | | | | | |
| 3 | Shoulder | 45′-90′ | +/−12 cm | 6 cm/sec | 15 cm | 5 cm | N/A |
| | Flexion | | | | | | |
| | 45′-90′ | | | | | | |
| 4 | Shoulder | 90′-112′ | +/−17 cm | 12 cm/sec | 5 cm | 5 cm | N/A |
| | Flexion | | | | | | |
| | 90′-120′ | | | | | | |
| 5 | Shoulder | N/A | +/−15 cm | N/A | 4 cm | 4 cm | N/A |
| | Stasis— | | | | | | |
| | Active | | | | | | |
| 6 | Shoulder | 112′-90′ | +/−8 cm | 5 cm/sec | 4 cm | 5 cm | 1.8 secs |
| | Extension | | | | | | |
| | 120′-90′ | | | | | | |
| 7 | Shoulder | 90′-45′ | +/−8 cm | 6 cm/sec | 5 cm | 4 cm | N/A |
| | Extension | | | | | | |
| | 90′-45′ | | | | | | |
| 8 | Shoulder | 45′-0′ | +/−6 cm | 17 cm/sec | 4 cm | 4 cm | N/A |
| | Extension | | | | | | |
| | 45′-0′ | | | | | | |

TABLE 7
Measured ranges of movement and coordination and cognition
characteristics for elbow elementary movements

| | | | Coordination | | | | |
|---|---|---|---|---|---|---|---|
| | | | | | Posterior | Lateral | |
| | Elementary | Measured | Movement | | Trunk | Trunk | |
| Step | movement | range | Precision | Speed | Compensation | Compensation | Cognition |
| 2 | Elbow | 60′-120′ | +/−8 cm | 6 cm/sec | 25 cm | 5 cm | 0.7 secs |
| | Extension | | | | | | |
| | 60′-160′ | | | | | | |
| 3 | Elbow | 120′-60′ | +/−8 cm | 8 cm/sec | 15 cm | 4 cm | N/A |
| | Flexion | | | | | | |
| | 160′-60′ | | | | | | |
| 4 | Forearm | 60′-100 | +/−6 cm | 11 cm/sec | 5 cm | 5 cm | N/A |
| | Rotation | | | | | | |
| | 60′-100′ | | | | | | |

If a series of movement sequences is performed, endurance characteristics may be calculated by the evaluation module 204. For example, the endurance characteristics may comprise a movement consistency and a trunk compensation. It should be understood that target values or target range of values are then included in the sequence of target movements and therefore received by the evaluation module 204. Table 8 presents exemplary endurance characteristics for the elbow elementary movements.

TABLE 8
Endurance characteristics for elbow elementary movements.

| | |
|---|---|
| Component Tasks | Endurance |

| Sequence | (B) | Movement | Trunk |
|---|---|---|---|
| Steps | (Elbow) | Consistency | Compensation |
| 2 | Elbow Extension | 18% precision | 16% increase |
| | 60′-160′ | lost | |
| 3 | Elbow Flexion | 20% precision | 22% increase |
| | 160′-60′ | lost | |
| 4 | Forearm Rotation | 17% precision | 27% increase |
| | 60′-100′ | lost | |

Then the evaluation module 204 compares the measured range of movement of each elementary movement to its corresponding target range of movement. The evaluation module 204 may attributes a comment to each elementary movement such as “successful” or “unsuccessful” to each elementary movement based on the result of the comparison. In the same or another embodiment, the evaluation module 204 may determine a percentage of accomplishment for each elementary movement, as illustrated in tables 9 and 10. The evaluation module 204 further compares the determined coordination characteristics to their respective target value or target range, and may attribute comments based on the comparison. For example, the evaluation module 204 may characterize a determined speed of movement as slow when below a first threshold, as medium when comprised between the first threshold and a second threshold, and as high when above the second threshold. It should be understood that the first and second threshold correspond the target range for the speed of movement, which is included in the target sequence of movement.

TABLE 9
Comparison results for the shoulder elementary movements

| | | | Coordination | |
|---|---|---|---|---|
| | Elementary | Movement | Movement | |
| Step | movement | Accomplished | Precision | Speed |
| 1 | Shoulder Abduction | 100% | Satisfactory | Slow |
| | 0-15′ | | | |
| 2 | Shoulder Flexion | 100% | Borderline | Slow |
| | 0-45′ | | Satisfactory | |
| 3 | Shoulder Flexion | 100% | Non-satisfactory | Slow |
| | 45′-90′ | | | |
| 4 | Shoulder Flexion | 73% | Non-satisfactory | Medium |
| | 90′-120′ | | | |
| 5 | Shoulder Stasis - | N/A | Non-satisfactory | N/A |
| | Active | | | |
| 6 | Shoulder Extension | 73% | Borderline | Slow |
| | 120′-90′ | | Satisfactory | |
| 7 | Shoulder Extension | 100% | Borderline | Slow |
| | 90′-45′ | | Satisfactory | |
| 8 | Shoulder Extension | 100% | Satisfactory | Medium |
| | 45′-0′ | | | |

| | | Coordination | | Cognition |
|---|---|---|---|---|
| | Elementary | Posterior Trunk | Lateral Trunk | Reaction |
| Step | movement | Compensation | Compensation | Time |
| 1 | Shoulder Abduction | OK | OK | Slow |
| | 0-15′ | | | |
| 2 | Shoulder Flexion | Too much | OK | N/A |
| | 0-45′ | | | |
| 3 | Shoulder Flexion | Too much | OK | N/A |
| | 45′-90′ | | | |
| 4 | Shoulder Flexion | OK | OK | N/A |
| | 90′-120′ | | | |
| 5 | Shoulder Stasis - | OK | OK | N/A |
| | Active | | | |
| 6 | Shoulder Extension | OK | OK | Medium |
| | 120′-90′ | | | |
| 7 | Shoulder Extension | OK | OK | N/A |
| | 90′-45′ | | | |
| 8 | Shoulder Extension | OK | OK | N/A |
| | 45′-0′ | | | |

TABLE 10
Comparison results for the elbow elementary movements

| | Elementary | Movement | Coordination | |
|---|---|---|---|---|
| Step | movement | Accomplished | Movement Precision | Speed |
| 2 | Elbow Extension | 60% | Borderline | Slow |
| | 60′-160′ | | Satisfactory | |
| 3 | Elbow Flexion | 60% | Borderline | Slow |
| | 160′-60′ | | Satisfactory | |
| 4 | Forearm Rotation | 100% | Satisfactory | Medium |
| | 60′-100′ | | | |

| | | Coordination | | Cognition |
|---|---|---|---|---|
| | Elementary | Posterior Trunk | Lateral Trunk | Reaction |
| Step | movement | Compensation | Compensation | Time |
| 2 | Elbow Extension | Too much | OK | Fast |
| | 60′-160′ | | | |
| 3 | Elbow Flexion | Too much | OK | N/A |
| | 160′-60′ | | | |
| 4 | Forearm Rotation | OK | OK | N/A |
| | 60′-100′ | | | |

When it is adapted to determine endurance characteristics, the evaluation module 204 compares the determined endurance characteristics to target values or target ranges, which are included in the received sequence of movement. The evaluation module 204 then assigns a comment to each endurance characteristic based on the comparison, as illustrated in Table 11.

TABLE 11
Comparison results for the elbow endurance characteristics

Endurance

| | | Movement | Trunk |
|---|---|---|---|
| Step | (Elbow) | Consistency | Compensation |
| 2 | Elbow Extension | Unsatisfactory | Borderline |
| | 60′-160′ | | satisfactory |
| 3 | Elbow Flexion | Unsatisfactory | Unsatisfactory |
| | 160′-60′ | | |
| 4 | Forearm Rotation | Borderline | Unsatisfactory |
| | 60′-100′ | satisfactory | |

In one embodiment, the target values or target ranges for the additional characteristics such as the coordination, cognition, and endurance characteristics are stored in the database of the medical professional machine or the server which automatically retrieve and include them in the sequence of target elementary movements. It should be understood that the medical professional may access the target values or target range and modify them.

In another embodiment, the target values or target ranges for the additional characteristics are not included in the sequence of elementary movements but stored locally on the evaluation module 204.

In a further embodiment, the target values or target ranges may correspond to thresholds which may be generated by the medical professional machine or the server, by interpolating between two sets of data stored in the database. The first set of data comprises the patient's assessment data on the relevant characteristics, which was measured at the start of the patient's rehabilitation program. The second set of data comprises a set of performance milestones that a clinician aims to see the user reaching during the course of the rehabilitation program. The exact threshold values may be generated based on a function of time elapsed from when the original assessment data was taken, and the milestones dates. The function of interpolation may be linear or non-linear. This thresholding process may segment the qualitative evaluation into two or more levels such as “satisfactory” when the determined characteristic is below a first threshold, “borderline satisfactory” when the determined characteristic is between the first threshold and a second threshold, and “unsatisfactory” when the determined characteristic is above the second threshold.

In one embodiment, the evaluation module 204 is further adapted to correlate specific movement deficiencies with controlling muscles for greater clarity of information to the supervising clinician. Problematic issues regarding muscles, joints, nerves, bones, or particular pathologies are identified by analyzing specific deficiencies as reflected in the comparison results.

For example, shoulder flexion at a certain angle range may require a certain set of muscles in order to perform, while shoulder flexion at another angle range may require another set of muscles in order to perform. Therefore, if the user cannot successfully perform shoulder flexion at a first range of angle, the cause of the failure may be related to a pathology of a first set of muscles. If the user cannot successfully perform shoulder flexion at a second and different range of angle, the cause of the failure may be related to a pathology of a second and different set of muscles.

The evaluation module 204 comprises a database in which combinations of an elementary movement and a movement range are stored. For each combination contained in the database, the database further comprises an identification of a muscle, a set of muscles, a joint, a set of joints, a nerve, a set or nerves, a bone, a set of bones, and/or the like that may be responsible for the failure of the user to adequately execute the corresponding elementary movement. The database may further comprise an identification of a pathology for at least one combination contained in the database.

This way, the system can match patterns of movement in patient performance to known clinical syndromes. It does this by comparing data on patient performance against reference data in a database of data and data relationships found in specific clinical syndromes and clinical deficiencies.

For example, in the database, disorders of cervical nerve C5 (e.g. compression by cervical disk or osteophytes), as well as disorders of the supraspinatus muscle (e.g. surpraspinatus muscle tear) may be associated with lateral abduction of the arm. Disorders of cervical nerve C6, as well as disorders of the brachioradialis muscle may be associated with elbow flexion. Disorders of cervical nerve C7, as well as disorders of the triceps muscle may be associated with elbow extension.

Therefore, if it determines that the user faded to adequately execute an elbow extension, the evaluation module 204 retrieves the corresponding possible causes for the failure, i.e. cervical nerve C7 and triceps muscle, and includes them in the outputted evaluation report.

In one embodiment, after identifying the elementary movements that the user failed to adequately execute, the evaluation module 204 verifies if the identified elementary movements are part of rehabilitation goals or priorities for the user. In this case, the evaluation module 204 receives combinations of elementary movements and corresponding movement ranges, which correspond to rehabilitation priorities for the user. Table 12 illustrates a list of rehabilitation priorities for a given user. If an elementary movement identified that the user failed to adequately execute is not included in the list of rehabilitation priorities, then the evaluation unit 204 does not include the given elementary movement in the evaluation report or considers it as satisfactory. The evaluation module 204 may only consider as unsatisfactory the elementary movements which are failed by the user and included in the list of rehabilitation priorities.

TABLE 12
Rehabilitation priority list
Rehabilitation Goals

Shoulder Abduction 0-120′
Shoulder Flexion 0-100′
Shoulder Extension 120′-0′
Elbow Flexion 0-90′
Forearm Internal Rotation 0′-100′
Trunk Rotation 0-90′

In an embodiment in which a virtual user-controlled object is to be controlled by the hand of the user during the interactive simulation, the 3D position of a given point of the hand such as the position of the palm, or the position of the wrist of the user may be tracked and used for moving the virtual user-controlled object. This may be the case when the target elementary movements include an elbow movement of a shoulder movement. For example, an activity focusing on the training of elbow extension may require the user to move his hand from a near point to a further point in depth to move a virtual user-controlled object, thus performing an elbow extension. However, in this case, the patient can still achieve a successful activity result without performing the elbow extension, and instead by compensating his movement by leaning his trunk forward. In fact the user may successfully move the virtual user-controlled object within the simulation while moving his trunk and not executing the elbow extension. In this case, the virtual user-controlled object of which the position is controlled by the position of the wrist will be adequately moved within the simulation, giving the user a false visual indication that he successfully performs while he is not adequately performing the elbow extension.

In one embodiment, the above-identified problem may be overcome by controlling the depth position of the user-controlled object using the elbow angle, i.e. the angle between the forearm and the arm. In this case, the position of the virtual user-controlled object within the simulation is controlled as a function of the elbow angle. Therefore, if the user moves his trunk instead of extending his elbow, the position of the user-controlled object will not be changed, thereby giving the user a visual indication that he does not perform the right movement. It should be understood that the shoulder angle may be used for controlling a virtual user-controlled object during a rehabilitation exercise requiring a shoulder movement in order to avoid any trunk compensation from providing the user with wrong visual indication during the simulation.

In another embodiment, the position of the user-controlled object may be controlled using the distance between the hand or the wrist and the trunk or the chest of the user. In this case, the position of the virtual user-controlled object within the simulation is controlled as a function of the distance. Therefore, if the user moves his trunk instead of extending his elbow, the position of the user-controlled object will not be changed, thereby giving the user a visual indication that he does not perform the right movement.

In one embodiment, no instructions are provided to the user for executing the task in the simulation. For example, the task to be executed by the user in the simulation may be implicit so that the user understands the task to be executed from the displayed simulation.

In another embodiment, instructions are provided to the user in order to explain to the user the given task that he should accomplish in the simulation. It should be understood that the instructions may be visual instructions, oral instructions, written instructions, etc. For example, a text may be inserted in the displayed simulation in order to inform the user of the task to be executed. Virtual objects of the simulation may also be used to provide visual instructions to the user. For example, arrows may provide the user with a movement direction for moving his hand.

While in the present description, the simulation generator 202 receives position information to change the characteristic of the virtual user-controlled object from the motion sensing device 206, and the evaluation module 204 receives the measured elementary movements from the motion sensing device 206, it should be understood that other configurations are possible.

In an embodiment in which the simulation generator 202 is adapted to modify the characteristic of the virtual user-controlled object using the measured elementary movements, the measured elementary movements may be transmitted from the motion sensing device 206 to the simulation generator 202 which transmits them to the evaluation module 204. Alternatively, the measured elementary movements may be transmitted from the motion sensing device 206 to the evaluation module 204 which transmits them to the simulation generator 202.

In an embodiment in which the simulation generator 202 is adapted to modify the characteristic of the virtual user-controlled object using position information other than the measured elementary movements, such as the 3D position of a wrist when the measured elementary movements comprise an elbow flexion and a should adduction, the measured elementary movements and the addition position information may be transmitted from the motion sensing device 206 to the simulation generator 202 which transmits the measured elementary movements to the evaluation module 204. Alternatively, the measured elementary movements and the addition position information may be transmitted from the motion sensing device 206 to the evaluation module 204 which transmits them to the simulation generator 202.

It should be understood that the simulation generator 202 and the evaluation module 204 may be part of a same device, apparatus, or system comprising at least a processing unit, a storing unit, and a communication unit for receiving and sending data. In another embodiment, the simulation generator 202 and the evaluation module 204 may be independent and each provided with at least a processing unit, a storing unit, and a communication unit for receiving and sending data. For example, the simulation generator 202 may be integrated in the user machine while the evaluation module 204 may be integrated in the medical professional machine or the server.

It should be understood that any adequate motion sensing device may be used such as an infrared motion sensing device, an optics motion sensing device, a radio frequency energy motion sensing device, a sound motion sensing device, a vibration motion sensing device, a magnetism motion sensing device, etc. In one example, the motion sensing device 206 may comprise a Kinect™.

It should be understood that the medical professional machine and the user machine may be integral together to form a single machine on which the interactive environment, the simulation, and the evaluation are generated.

In the following, different examples of rehabilitation exercises are presented.

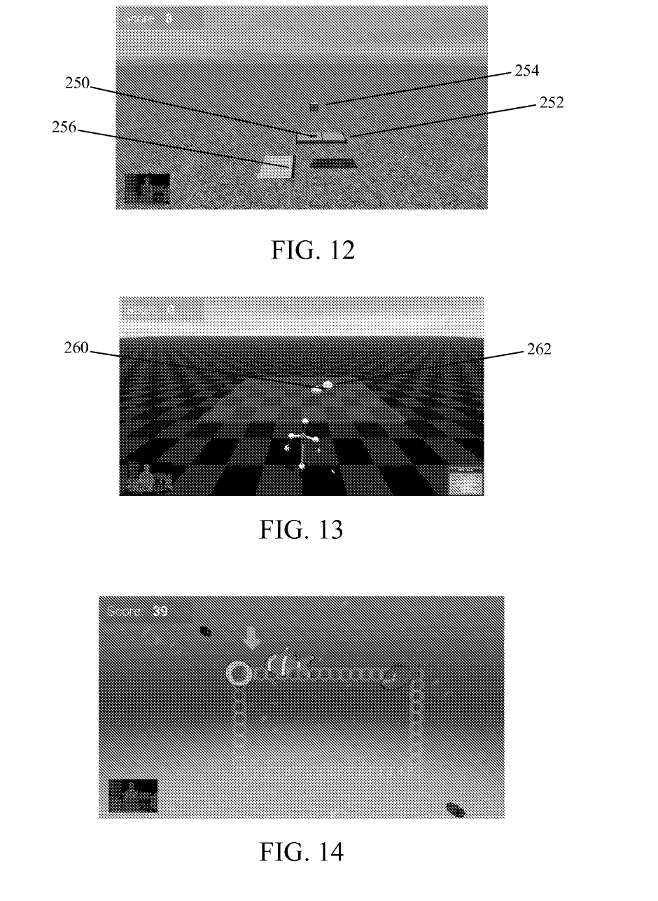

A first example consists in a rehabilitation exercise focusing on shoulder and elbow bilateral movements. Table 13 presents the sequence of elementary movements to be executed by the user during the rehabilitation exercise. The interactive environment comprises two platforms 250 and 252, a cubic object 254, and a basket 256, as illustrated in FIG. 12. It should be understood that the characteristics of the objects such as the shape, size, speed, if applicable, color, and/or the like, are defined in the interactive environment. It should also be understood that the position of the objects within the interactive environment is chosen as a function of the elementary movements to be executed by the user.

TABLE 13
Sequence of elementary movements for bilateral shoulder and elbow
training.

| | Simple Movement | Simple Movement | Simple Movement | Simple Movement |
|---|---|---|---|---|
| Step | Tasks (A) | Tasks (B) | Tasks (C) | Tasks (C) |
| 1 | Shoulder External | Shoulder Internal | Elbow Flexion 0′-90′ | Elbow Flexion 0′-60′ |
| | Rotation 90′-100′ | Rotation 90′-45′ | | |
| 2 | Shoulder Stasis | Shoulder Stasis | | |
| 3 | Shoulder Internal | Shoulder External | Elbow Flexion 90′-60′ | Elbow Flexion |
| | Rotation 100′-45′ | Rotation 45′-100′ | | 60′-90′ |
| 4 | Shoulder External | Shoulder External | | |
| | Rotation 45′-90′ | Rotation 100′-130′ | | |

During the simulation, the task of the patient consists in controlling the platforms 250 and 252. The horizontal position of the platform 250 is controlled by the position of the left hand of the user, and the horizontal position of the platform 252 is controlled by the position of the right hand of the user. When the user brings his two hands together, the two platforms 250 and 252 abut together and form a single extended platform. The user can then catch the cubic object 254 that falls from the top. The user cannot catch the cubic object 254 if his two hands are not brought together. Once caught, the user has to move the cubic object 254 on top of the basket 256 while maintaining his hands together, and release the cubic object 254 in the basket 256 by opening his hands apart.

In another example, a rehabilitation exercise focuses on the balance of a user. Table 14 presents the sequence of elementary movements to be executed by the user. An exemplary interactive environment adapted to allow the user perform the sequence of elementary movements presented in Table 14 is illustrated in FIG. 13. The interactive environment comprises a ball 260 and four target objects of which only target object 262 is illustrated in FIG. 13. The position of the target object is chosen as a function of the sequence of elementary movements to be executed by the user.

TABLE 14
Sequence of elementary movements for balance training.

| | Elementary Movement | Elementary Movement |
|---|---|---|
| Step | Tasks (A) | Tasks (B) |
| 1 | Trunk Forward Flexion 0′-25′ | Trunk Lateral Flexion 0′-15′ |
| 2 | Trunk Forward Extension 25′-0′ | Trunk Lateral Extension 15′-0′ |
| 3 | Trunk Forward Flexion 0′-15′ | Trunk Lateral Flexion 0′-(-)20′ |
| 4 | Trunk Forward Extension 15′-0′ | Trunk Lateral Extension (-)20′-0′ |

In this trunk movement and posture activity, the user must control the position of the ball 260 using the angle of his/her trunk, in order to hit the target object at various horizontal and depth positions. When the simulation starts, only one of the four target objects is displayed to the user. Once the user hit the first target object, the first target object disappears and the second target object appears. It should be understood that the position of the first target object is chosen as a function of the elementary movements to be executed at step 1 of the sequence. The position of the second target object is chosen as a function of the elementary movements to be executed at step 2 of the sequence, etc.

The above exercise may be adapted so that the ball 260 may be controlled by a hand of the user. In this case, the position of the target objects 262 may be changed for example.

In another example, a rehabilitation exercise may focus on a unilateral elbow and shoulder training with a speed component. Table 1 illustrates an exemplary sequence of elementary movements for such a training. An interactive environment corresponding the sequence of elementary movements presented in Table 15 is illustrated in FIG. 14. The interactive environment comprises a fish, a piranha, food object positioned according to a rectangle, a start indicator for indicating to the user the starting point, and an arrow for indicating the direction of movement to the user.

In this unilateral movement activity, the patient controls the position of the fish via the position of his right hand for example. The user is required to control the fish so that the fish eats the food objects starting from the start indicator and according to the direction illustrated by arrow. The user must accomplish this while keeping their elbow straight. Furthermore, the fish must avoid going below a certain speed in order to avoid getting eaten by the piranha.

TABLE 15
Sequence of elementary movements for a unilateral shoulder and
elbow training.

| | Elementary Movement | Elementary Movement |
|---|---|---|
| Step | Tasks (A) | Tasks (B) |
| 1 | Shoulder Forward Flexion 0′-60′ | Elbow Extension 90′-0′ |
| 2 | Shoulder Horizontal Abduction 90′-60′ | Elbow Stasis |
| 3 | Shoulder Forward Flexion 60′-120′ | |
| 4 | Shoulder Horizontal Adduction 60′-120′ | |
| 5 | Shoulder Forward Flexion 120′-60′ | |
| 6 | Shoulder Horizontal Abduction 120′-90′ | |

Although the above description relates to specific embodiments as presently contemplated by the inventors, it will be understood that the invention in its broad aspect includes hardware and functional equivalents of the elements described herein. Moreover, although the invention has been described in a particular application, it should be understood that the invention may be used in various other applications.

What is claimed is:

1. A computer-implemented method for evaluating a user during a virtual-reality rehabilitation exercise, comprising:
receiving a target sequence of movements comprising at least a first target elementary movement, and a second target elementary movement, the first target elementary movement defined by a first body part and a first movement type and the second target elementary movement defined by a second body part and a second movement type, the first and second target elementary movements being different;
receiving a measurement of a movement executed by the user while performing the rehabilitation exercise and interacting with a virtual-reality simulation comprising at least a virtual user-controlled object, a characteristic of the virtual user-controlled object being controlled by the movement;
determining, from the measurement of the movement executed by the user, a sequence of measured movements comprising at least a first measured elementary movement and a second measured elementary movement;
comparing the sequence of measured movements to the sequence of target movements, thereby obtaining an evaluation of a performance of the user; and
outputting the evaluation.

2. The computer-implemented method of claim 1, wherein said receiving a target sequence of movements further comprises receiving a first target range of movement for the first target elementary movement and a second target range of movement for the second target elementary movement, and said determining a sequence of measured movements further comprises determining a first measured range of movement for the first target elementary movement and a second measured range of movement for the second target elementary movement, at least one of the first and second target elementary movements being different and the first and second target ranges of movement being different.

3. The computer-implemented method of claim 2, wherein said receiving the measurement of the movement executed by the user comprises receiving the first measured range of movement for the first measured elementary movement and the second measured range of movement for the second measured elementary movement, and said determining the sequence of measured movements comprises temporally ordering the first and second elementary movements.

4. The computer-implemented method of claim 2, wherein said receiving the measurement of the movement executed by the user comprises receiving position information for the first body part and the second body part.

5. The computer-implemented method of claim 4, wherein said receiving position information comprises, for each one of the first and second body parts, receiving at least one of an angle in time and a position in time of reference points.

6. The computer-implemented method of claim 5, wherein said determining a sequence of measured elementary movements comprises:
determining, for each one of the first and second body parts, a type of movement, thereby identifying the first and second measured elementary movements;
determining the first and second measured range of movements from the position information; and
determining an order of execution for the first and second measured elementary movements, thereby obtaining the sequence of measured elementary movements.

7. The computer-implemented method of claim 6, wherein said determining a type of movement comprises:
determining a movement axis from the position information; and
assigning the type of movement as a function of the movement axis.

8. The computer-implemented method of claim 7, wherein said determining a type of movement further comprises determining a movement direction using the position information, said assigning the type of movement comprising assigning the type of movement as a function of the movement axis and the movement direction.

9. The computer-implemented method of claim 2, wherein said comparing comprises:
comparing the first and second measured elementary movements to the first and second target elementary movements, respectively; and
comparing the first and second measured ranges of movement to the first and second target ranges of movement, respectively.

10. The computer-implemented method of claim 1, wherein said outputting an evaluation comprises outputting an indication as to whether the user failed to execute at least one of the first and second target elementary movement.

11. A system for evaluating a user during a virtual-reality rehabilitation exercise, comprising:
a communication unit for:
receiving a target sequence of movements comprising at least a first target elementary and a second target elementary movement, the first target elementary movement defined by a first body part and a first movement type and the second target elementary movement defined by a second body part and a second movement type, the first and second target elementary movements being different;
receiving a measurement of a movement executed by the user while performing the rehabilitation exercise and interacting with a virtual-reality simulation comprising at least a virtual user-controlled object, a characteristic of the virtual user-controlled object being controlled by the movement; and
outputting an evaluation of a performance of the user;
a sequence determining unit for determining, from the measurement of the movement executed by the user, a sequence of measured movements comprising at least a first measured elementary movement and a second measured elementary movement; and
a comparison unit for comparing the sequence of measured movements to the sequence of target movements in order to obtain the evaluation of the performance of the user.

12. The system of claim 11, wherein the communication unit is further adapted to receive a first target range of movement for the first target elementary movement and a second target range of movement for the second target elementary movement, and the sequence determining unit is further adapted to determine a first measured range of movement for the first target elementary movement and a second measured range of movement for the second target elementary movement, at least one of the first and second target elementary movements being different and the first and second target ranges of movement being different.

13. The system of claim 12, wherein the measurement of the movement executed by the user comprises the first measured range of movement for the first measured elementary movement and the second measured range of movement for the second measured elementary movement the sequence determining unit is adapted to temporally order the first and second elementary movements.

14. The system of claim 12, wherein the measurement of the movement executed by the user comprises position information for the first body part and the second body part.

15. The system of claim 14, wherein the position information comprises, for each one of the first and second body parts, at least one of an angle in time and a position in time of reference points.

16. The system of claim 15, wherein the sequence determining unit is adapted to:
determine, for each one of the first and second body parts, a type of movement, thereby identifying the first and second measured elementary movements;
determine the first and second measured range of movements from the position information; and
determine an order of execution for the first and second measured elementary movements in order to obtain the sequence of measured elementary movements.

17. The system of claim 16, the sequence determining unit is further adapted to:
determine a movement axis from the position information; and
assign the type of movement as a function of the movement axis.

18. The system of claim 17, wherein the sequence determining unit is further adapted to determine a movement direction from the position information and assign the type of movement as a function of the movement axis and the movement direction.

19. The system of claim 12, wherein the comparison unit is adapted to:
compare the first and second measured elementary movements to the first and second target elementary movements, respectively; and
compare the first and second measured ranges of movement to the first and second target ranges of movement, respectively.

20. The system of claim 11, wherein the evaluation comprises an indication as to whether the user failed to execute at least one of the first and second target elementary movement.
